(19) United States
(12) Patent Application Publication
(10) Pub. No.: US 2013/0035704 A1
(43) Pub. Date: Feb. 7, 2013

(54) SURGICAL SPREADABLE SHEET DELIVERY AND POSITIONING SYSTEM AND METHOD

(71) Applicant: Moshe Dudai, Tel-Aviv (IL)

(72) Inventor: Moshe Dudai, Tel-Aviv (IL)

(21) Appl. No.: 13/641,149

(22) PCT Filed: Apr. 14, 2011
(86) PCT No.: PCT/IL2011/000320

§ 371 Date: Oct. 15, 2012

Related U.S. Application Data
(60) Provisional application No. 61/323,933, filed on Apr. 14, 2010.

(51) Int. Cl. A61B 17/11

(52) U.S. Cl. 606/151

(57) ABSTRACT

A prepackaged mesh unit which comprises a spreadable sheet, a guide rod configured for remotely positioning the spreadable sheet within a body cavity, and a plurality of self-extending elements collapsed with the spreadable sheet and held at one end by the guide rod, wherein the self-extending elements have an elastic property and store elastic energy while collapsed with the spreadable sheet, wherein the number and configuration of the self-extending elements is selected to match a size and shape of the spreadable sheet.

### FIELD OF THE INVENTION

The present invention relates to delivery and positioning of a surgical patch or the like and more particularly, but not exclusively, surgical spreadable sheet delivery and positioning for minimally invasive hernioplasty procedures.

### BACKGROUND OF THE INVENTION

A known technique for hernia repair is to place a prosthetic mesh over an effected area. The mesh is either placed over the defect (anterior repair) or under the defect (posterior repair). Staples may be used to keep the mesh in place. Mesh repair of hernias is sometimes referred to as “Tension Free” repairs because, unlike older more traditional methods, muscle surrounding the hernia is not pulled together and sutured under tension. The mesh approach to hernia repair avoids over-stretching the surrounding muscle which is often already weakened. Hernias can often be repaired in minimally invasive outpatient procedures. Known outpatient procedures for hernia repair include laparoscopic surgery, trans-abdominal procedures and natural orifice trans-abdominal endoscopic surgery. Mesh repair is also performed as part of post-operative procedures involving open surgery.

International Patent Application Publication WO2006/082587 entitled “Surgical Mesh, Mesh Introducing and Placing Devices and Methods”, the contents of which is incorporated herein by reference, describes an insertion device and method for inserting a surgical mesh into an abdominal cavity. Prior to insertion, the mesh together with one or more self-spreading elements is rolled from two opposite ends toward each other to form a double cylindrical scroll. The spreading elements originate and/or are connected to one end of leading rod which is positioned at a center of the mesh. The leading rod is used to control insertion and positioning of the mesh within abdominal cavity. Typically, the connection between the leading rod and the spreading elements is flexible allowing an operable range of motion of the leading rod. The spreading elements are typically elastic elements that will self-unravel when released. Fasteners are used to avoid unraveling of the mesh prior to insertion. Typically, the fasteners provide for separately unraveling each end of the mesh.

It is described that the mesh is introduced into the abdominal cavity through a relatively large diameter trocar that may typically be used for a scope. Insertion of the mesh is provided by connecting the leading rod to a hand guiding unit and directing the mesh into the abdominal cavity with the hand guiding unit. It is described that once the mesh is inserted, the leading rod is released from the hand guiding unit and the hand guiding unit is removed from the trocar so that a scope can be reintroduced through the trocar. The hand guiding unit without the mesh can be reintroduced through an alternate port site (including a trocar that is typically smaller) and reconnected to the leading rod for proper positioning of the mesh with visual aid. Once positioned, each end of the mesh can be separately released by releasing the fasteners and fixed to the abdominal wall.

International Patent Application Publication WO2009/104182 entitled “A Device and Method for Deploying and Attaching a Patch to a Biological Tissue,” describes an integrated deployment and attachment device (DAD) having a distal portion, adapted to be inserted into a body and a proximal portion, located adjacent to a user, the distal and proximal portions are interconnected along a main longitudinal axis via a tube. The tube accommodates a portion of a central shaft protruding from a distal end of the tube. The central shaft is adapted to reciprocally move parallel to the main longitudinal axis. The distal portion of the shaft includes two flexible arms adapted to be reversibly coupled to said patch. The two flexible arms are jointly connected at two ends and are characterized by having an initial stage at which the flexible arms are straight and parallel to the longitudinal axis of said central shaft; and, a final stage at which the flexible arms are laterally curved with respect the longitudinal axis of the central shaft such that the patch is deployed. The flexible arms further include attachment clips to attach the patch to the biological tissue and a connecting mechanism to reversibly connect the patch to the flexible arms. A handle in communication with the shaft and located outside said body is used to reversibly transform the flexible arms from the initial stage to the final stage.

US Patent Publication No. 20070260179 entitled “Hernia Repair Device,” the contents of which is incorporated herein by reference, describes an apparatus for treating hernia that includes an elongated open-bored applicator insertable via the hernia into the abdominal cavity; a collapsible mesh with a plane body rolled in the applicator and at least partially enveloped by one or more elastic collars, and a device for pushing said mesh throughout the open bore. It is described that the mesh is adapted to be deployed helically when ejected outside the applicator.

US Patent Publication No. 20070185506 entitled “Hernia Repair Device,” the contents of which is incorporated herein by reference, describes a medical instrument used to position a surgical mesh sheet to a surface of the human body through a surgical port. The instrument contains a number contact mechanism for holding the mesh sheet against the tissue surface, and a grasping mechanism for grasping and then quickly and easily releasing the mesh sheet from the instrument once it is in place. The contact mechanism is stored in a compressed form in the instrument. During deployment, the contact mechanism is pushed forward and expands out of the instrument until it contacts a mesh sheet that has already been placed against an abdominal wall. If the mesh sheet has been pre-loaded in the instrument, the grasper mechanism grasps an approximate center of the mesh while the contact mechanism and the attached mesh sheet is activated to open against the abdominal wall or other body surface. Further activation of the instrument releases the mesh from grasper mechanism. Optionally, the contract mechanism comprises an umbrella-like structure with any shape (e.g., circular, square, polygonal, etc.). After the mesh sheet is attached, the contact mechanism is retracted into an inner housing of the instrument, thereby allowing removal of the instrument from a port through which it was inserted.

U.S. Pat. No. 6,099,518 entitled “Needle Herniorrhaphy Devices,” the contents of which is incorporated herein by reference, describes a device for creating an operating space in registry with the herniated region of a patient and to deliver a surgical prosthetic mesh to the operating space created. The mesh is rolled in a single cylindrical scroll around a guide-wire conduit positioned through an elongated tubular mesh delivery device. Unraveling of the rolled mesh can be provided by an expandable balloon attached and rolled with the mesh or by prongs introduced through distal port holes and that latch on to two ends of the mesh and serve to unroll the mesh by a an exerted pull force. Optionally the mesh is folded once prior to rolling it into a single cylindrical pre so that both ends of the mesh can be pulled by prongs from opposite ends.

U.S. Pat. No. 5,405,360 entitled “Resilient arm mesh deployer,” the contents of which is incorporated herein by reference, describes an apparatus for positioning surgical implants adjacent to body tissue. The apparatus comprises an elongated rod having a plurality of delivery arms secured to distal end of a surgical implant for releasably receiving a peripheral portion of a surgical implant, securing means in contact with distal end of said delivery arms for detachably securing said surgical implant to said delivery arms, and a pusher rod secured to the distal end of said elongated rod for contacting and affecting tile shape of the surgical implant. Upon deployment, the delivery arms cause the peripheral portion of the surgical implant to expand and the pusher rod contacts the surgical implant at an interior portion of surgical implant spaced from the peripheral portions and affects the shape of said interior portion of said surgical implant.

### SUMMARY OF THE INVENTION

An aspect of some embodiments of the invention is the provision of systems and methods for surgical spreadable sheet, such as any surgical mesh, a mesh, a therapeutic film, a diagnostic film and the like (for brevity referred to herein as a spreadable sheet or a mesh) delivery and positioning. According to some embodiments of the present invention, the delivery and positioning system is a modular system that can be adapted to a particular hernia repair procedure and to particular mesh geometry required for the procedure. According to some embodiments of the present invention, the system includes a prepackaged mesh unit including a mesh packaged with at least one self-extending element engaging the mesh and connected to a guide rod. According to some embodiments of the present invention, the self-extending elements connected to the guide rod are selected for a particular application and/or mesh geometry. In some exemplary embodiments, the length of the guide rod is selected to correspond with the size of the mesh when collapsed in the prepackaged mesh unit. According to some embodiments of the present invention, the mesh is packaged in the prepackaged mesh unit so that a tip of the guide rod connected to the self extending element is positioned in the center of the mesh and thereby marks the center of the mesh. In some exemplary embodiments, the systems and methods provide for centering the mesh with respect to the herniated area while the mesh is at least partially collapsed in the prepackaged mesh unit so that visibility of the hernia is maintained during centering of the mesh with respect to the hernia.

According to some embodiments of the present invention the prepackaged mesh unit including a mesh folded in a double cylinder scroll fold and self-extending unit positioned in between the cylinders. Typically, a restricting element restricts extension of the self extending unit and upon releasing of the restricting element, the self-extending unit spreads itself and the mesh.

An aspect of some embodiments of the present invention provides for a modular surgical mesh delivery and positioning system comprising: a prepackaged mesh unit, wherein the prepackaged mesh unit is configured for accommodating meshes of different size and shape, the prepackaged mesh unit including a mesh collapsed with a plurality of self-extending elements held by a guide rod, wherein the number and configuration of the self-extending elements is selected to match the mesh packaged in the prepackaged mesh unit; a hand guide unit operative to engage onto one end of the guide rod and thereby manipulate movement of the prepackaged mesh unit; and an operative channel adapted to be partially inserted in-vivo through which the prepackaged mesh unit is delivered, wherein each of the prepackaged mesh unit, the hand guide unit and the operative channel are separately adaptable to specific applications while still maintaining compatibility with each other.

Optionally, a length of the guide rod is selected to correspond to a length of the mesh packaged in the prepackaged mesh unit.

Optionally, a length of the hand guide unit to be used with the guide rod is selectable on-site.

Optionally, a diameter of the operative channel is adapted to a diameter of the prepackaged mesh unit.

Optionally, the diameter of the operative channel is smaller than a diameter of the prepackaged mesh unit and is adapted to compress the prepackaged mesh unit during delivery.

Optionally, a length of the operative channel is adapted to the length of the prepackaged mesh unit.

Optionally, the operative channel includes extensions for adjusting a length of the operative channel.

Optionally, the hand guide unit is adapted to controllably latch on to and release the guide rod.

An aspect of some embodiments of the present invention provides for a prepackaged mesh unit comprising: a mesh; a guide rod configured for remotely positioning the mesh unit within a body cavity; and a plurality of self-extending elements collapsed with the mesh and held at one end by the guide rod, wherein the self-extending elements have an elastic property and store elastic energy while collapsed with the mesh, wherein the number and configuration of the self-extending elements is selected to match a size and shape of the mesh.

Optionally, at least a portion of the self-extending elements are configured to be separately extended.

Optionally, each of the plurality of self-extending elements are removably attached to the mesh.

Optionally, the mesh includes a plurality of bands through which the self-extending elements are inserted.

Optionally, the bands form pockets.

Optionally, the bands are constructed from a material of the mesh.

Optionally, at least one self-extending element includes a rigid portion and a flexible portion.

Optionally, the plurality of self-extending elements includes restricting elements for restricting extension of the self-extending elements.

Optionally, the self-extending elements are constructed from wire forming a loop at an end distal to an end held by the guide rod.

Optionally, the wire forming the loop has a ridged surface.

Optionally, at least one self-extending element is constructed from at least two wires fitted through a rigid tube, wherein the at least two wires are partially exposed over a length to form an elastic joint.

Optionally, at least one self-extending element is constructed from at least one wire fitted through a rigid tube, wherein the at least one wire is partially exposed over a length and is formed in a coil over that length.

Optionally, the rigid tube includes at least one element for securing the self-extending element in a collapsed position.

Optionally, the prepackaged mesh unit includes a hand guide, and a user controlled element on the hand guide is adapted to control extension of the self-extending elements.

Optionally, at least a portion of the self-extending elements are connected to each other by a string or wire.

Optionally, the string or wire restricts distancing of the self-extending elements at an end distal to an end held by the guide rod.

Optionally, the mesh is rolled in a double cylinder scroll in the prepackaged mesh unit.

Optionally, wherein ends of the self-extending elements are attached to corners of the mesh and the corners of the mesh are folded toward the center of the mesh prior to rolling the mesh in a double cylinder scroll.

Optionally, the plurality of self-extending elements are adapted to be placed in between the scrolls of double cylinder scroll while collapsed and to unroll the mesh in response to releasing the self-extending elements.

Optionally, at least one of the plurality of self-extending elements is at least partially constructed from shape memory alloy.

Optionally, the shape memory alloy is 0.4-0.5 mm nickel titanium wire.

Optionally, the guide rode is connected to the self-extending elements through a holding element, wherein the holding element is connected to the guide rod via a hinged connection.

Optionally, the holding element includes a locking mechanism, wherein the locking mechanism maintains the self-extending elements in a first configuration adapted for delivery and mesh deployment while locked, and collapses the self-extending elements in a second configuration adapted for extraction of the self-extending elements while unlocked.

Optionally, the locking mechanism is controlled by a user controlled element on the hand guide unit.

Optionally, the guide rod includes a flexible joint.

Optionally, the mesh is a self-adhesive mesh and wherein the mesh is packaged with a protective cover adapted to prevent the mesh from sticking to itself while in a collapsed in the prepackaged mesh unit.

Optionally, the mesh is a horseshoe shaped mesh shaped with an indented area and wherein two of the elastic self-extending elements are removably connected to the mesh on either side of the indented area and are adapted to maintain a ‘V’ shaped angle in an unloaded state.

Optionally, the mesh is covered with a protective sheath, wherein the sheath is adapted to be removed as it is passed through the operative channel.

Optionally, the sheath is fixedly attached to the hand guide unit.

Optionally, the sheath provides a hermetic seal for the mesh.

An aspect of some embodiments of the present invention provides for a prepackaged mesh unit adapted to be delivered in-vivo comprising: a guide rod constructed from a rigid material, wherein the guide rod includes a first and second end, wherein the first end is exposed and adapted to be engaged during delivery and positioning of the mesh; a plurality of elongated elastic or super elastic elements connected to the second end of the guide rod, wherein the elastic elements extend away from the guide rod in an unloaded state and are bent back over the guide rod in a loaded state; a mesh positioned over the elastic elements while the elastic elements are bent back over the guide rod in a loaded state; and a locking mechanism adapted for separately maintaining at least two portions of the elastic elements in a loaded position and for separately releasing each of the two portions.

Optionally, the mesh includes a plurality of bands through which the elastic elements are inserted.

Optionally, the bands form pockets.

Optionally, the bands are constructed from a material of the mesh.

Optionally, the elastic elements are constructed from wire forming a loop at an end distal to an end held by the guide rod.

Optionally, the wire is formed from shaped memory alloy.

Optionally, the guide rod includes a flexible joint.

Optionally the unit comprises a tube extending along the guide rod and at least one self-extending element and adapted to be accessed from outside of the body cavity and through which biological glue is injected for fixating the mesh.

Optionally, the unit comprises a plurality of string elements connected to the mesh and extending along the guide rod, the strings adapted to provide holding the mesh from outside the body cavity and to pull the mesh toward a herniated area.

Optionally, the prepackaged mesh unit is adapted to be inserted through a hernia defect.

Optionally, the elongated elastic elements are further extended away from the guide rod and straightened while extracted from a body cavity through an operative channel.

An aspect of some embodiments of the present invention provides for a prepackaged mesh unit adapted to be delivered in-vivo comprising: a mesh packaged with one or more self-extending elements, wherein the self-extending elements have an elastic property and are packaged in a loaded state; a restricting element adapted to restrict unloading of the self-extending elements, wherein said restricting elements confines the prepackaged mesh unit to a defined storage volume; a compressing element adapted to reduce a volume occupied by the prepackaged mesh unit from a defined storage volume to a defined in-vivo delivery volume on-site prior to delivery.

Optionally, the compressing element includes a sheath enclosing the mesh, wherein the sheath includes a valve for removing air from a volume enclosed by the sheath to reduce the volume occupied by the prepackaged mesh unit on-site.

Optionally, the compressing element includes a string or band spirally wrapped around the mesh and wherein tension applied to the string or band effects reducing the volume occupied by the prepackaged mesh unit.

An aspect of some embodiments of the present invention provides for a prepackaged mesh unit adapted for a hiatal hernia repair procedure comprising: a horseshoe shaped mesh including an indented area shaped for positioning around the esophagus; and a guide rod including a plurality of elongated elastic self-extending elements that extend from one end of the guide rod, wherein two of the elastic self-extending elements are removably connected to the mesh on either side of the indented area and are adapted to maintain a ‘V’ shaped angle in an unloaded state, and wherein the mesh is rolled from two ends with the two elastic self-extending elements into a double cylinder scroll toward the indented area.

Optionally, ends of the self-extending elements are attached to corners of the mesh.

Optionally, the corners of the mesh are folded toward the center of the mesh prior to rolling the mesh from two ends in the double cylinder scroll toward the indented area.

Optionally, the self-extending elements include rigid portions and flexible elastic portions, the flexible elastic portions forming flexible self-extending joints between the rigid portions.

Optionally, the elastic portions are constructed from flexible wire.

Optionally, the wire is composed of shape memory alloy.

An aspect of some embodiments of the present invention provides for a prepackaged mesh unit comprising: a mesh rolled in a double cylinder scroll; and a self extending unit adapted to be placed in between the double cylinder of the scroll; at least one restricting element positioned around the self-extending unit for restricting extension of the self-extending unit, and wherein the self extending unit unrolls the mesh in response to releasing the at least one restricting element.

Optionally, the self extending unit is attached to a guide rod adapted manipulate movement of the prepackaged mesh unit, wherein the guide rod is adapted to engage with a hand guide, the hand guide adapted to manipulate the guide rod from outside the body cavity.

Optionally, the restricting elements are released by a release button on the hand guide.

Optionally, the self-extending unit is constructed from a plurality of wires fitted through rigid tubes, wherein the wires are partially exposed to form elastic joints in exposure areas.

Optionally, the self-extending unit is constructed from a wire fitted through rigid tubes, wherein the wire is partially exposed and constructed in a coil in the exposed area to form an elastic joint in the exposure area.

Optionally, the rigid tube includes opening through which the restricting element is fitted to restrict extension of the self-extending unit.

An aspect of some embodiments of the present invention provides for a method for delivering and positioning a surgical mesh in a normal orifice transendoscopic surgical procedure, the method comprising: connecting a prepackaged mesh unit to an end of a flexible tube of an endoscope, wherein the prepackaged mesh unit includes a mesh collapsed around a plurality of self-extending elements; guiding the prepackaged mesh unit to a herniated area; and releasing packaging of the mesh unit, said self-extending elements operative to flatten out the mesh.

Optionally, the method comprises releasing a first portion of the self-extending elements, the first portion adapted to deploy a first half of the mesh; centering the mesh with the herniated area while only the first half of the mesh is deployed; and releasing a second portion of the self-extending elements, the second portion adapted to deploy a second half of the mesh after the centering.

Optionally, the method comprises fixating the first half of the mesh after the centering and prior to deploying the second half of the mesh.

Optionally, the method comprises positioning the mesh on the herniated area by sliding the plurality of self-extending elements and mesh against the herniated area.

An aspect of some embodiments of the present invention provides for an endoscope with prepackaged mesh unit comprising: an endoscope including a channel through which a flexible rod is introduced; a prepackaged mesh unit attached to the tip of the flexible rod through which it is led into the body cavity, wherein the prepackaged mesh unit comprises a mesh and a plurality of self-extending elements collapsed with the mesh and held at one end by the flexible rod, wherein the self-extending elements have an elastic property and store elastic energy while collapsed with the mesh, a restricting element for holding the self-extending elements collapsed with the mesh.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A, 1B and 1C are simplified schematic illustrations of three basic units making up the modular surgical mesh delivery and positioning system in accordance with some embodiments of the present invention;

FIGS. 2A, 2B, 2C and 2D are illustrations of an exemplary guide rod including exemplary self-extending elements in an unloaded, loaded, fully radially extended position and fully longitudinally extended position in accordance with some embodiments of the present invention;

FIGS. 3A, 3B and 3C are simplified schematic illustrations describing an umbrella packaging configuration that may be used for collapsing a mesh in the prepackaged mesh unit in accordance with some embodiments of the present invention;

FIGS. 4A and 4B are simplified schematic illustrations of an exemplary guide rode including self-extending elements connected by flexible string or wire for expanding a packaged mesh in accordance with some embodiments of the present invention;

FIGS. 5A and 5B are simplified schematic illustrations of a mesh including exemplary restraining elements to hold the self-extending elements during self-expansion of the prepackaged mesh unit in accordance with some embodiments of the present invention;

FIGS. 6A and 6B are simplified schematic illustrations of two additional exemplary guide rods including exemplary self-extending elements for deploying a mesh in an umbrella fold in accordance with some embodiments of the present invention.

FIGS. 7A and 7B are simplified schematic illustration describing an exemplary double cylinder scroll packaging method that may be used for collapsing a mesh in the prepackaged mesh unit in accordance with some embodiments of the present invention;

FIGS. 8A and 8B are simplified schematic illustrations describing an exemplary double cylinder scroll packaging method that may be used for collapsing a mesh in the prepackaged mesh unit in accordance with some embodiments of the present invention;

FIGS. 9A and 9B are simplified schematic illustrations describing an exemplary horseshoe double cylinder scroll packaging method that may be used for collapsing a mesh in the prepackaged mesh unit in accordance with some embodiments of the present invention;

FIGS. 10A, 10B, and 10C are simplified schematic illustration of an exemplary self-extending unit adapted to be inserted into a partially rolled double cylinder scroll in accordance with some embodiments of the present invention;

FIGS. 11A and 11B are simplified schematic illustration of an exemplary self-extending element including string structures adapted to be inserted into a partially rolled double cylinder scroll in accordance with some embodiments of the present invention;

FIGS. 12A and 12B are simplified schematic illustrations of an exemplary guide rod with self-extending elements including locking mechanisms for controllably locking and releasing the self-extending elements in accordance with some embodiments of the present invention;

FIGS. 12C and 12D shows an a exemplary method for folding a rectangular mesh in accordance with some embodiments of the present invention;

FIGS. 12E and 12F shows an a exemplary method for maintaining a rectangular mesh in a collapsed state (folded and rolled) in accordance with some embodiments of the present invention;

FIGS. 12G-12I shows exemplary arrangements of a mesh that is provided in a kit with a device such as depicted in FIG. 12A in accordance with some embodiments of the present invention;

FIGS. 13A and 13B are simplified schematic illustrations of an exemplary guide rod with self-extending elements as positioned respectively in a delivery configuration and fully extended configuration for extraction through a delivery channel in accordance with some embodiments of the present invention;

FIGS. 13C-13G are simplified schematic illustrations of an exemplary guide rod with exemplary self-extending elements and an exemplary self-extending element which are set to be manufactured plates in accordance with some embodiments of the present invention;

FIGS. 14A and 14B are alternate configurations of extending elements adaptable for larger scale meshes in accordance with some embodiments of the present invention;

FIGS. 14C and 14D are simplified schematic illustrations of an exemplary guide rod with self-extending elements including a plurality of elastic joints on each of the self-extending elements shown in a spread and folded configuration respectively in accordance with some embodiments of the present invention;

FIG. 14E is a simplified schematic illustration of an exemplary guide rod with self-extending elements which are set to spread a mesh to adapt to the curvature of a non planner surface in accordance with some embodiments of the present invention;

FIGS. 15A and 15B are simplified schematic illustration of an exemplary self-extending unit adapted to be inserted into a partially rolled horseshoe mesh using a horseshoe double cylinder scroll method, shown respectively in a full extended and collapsed configuration in accordance with some embodiments of the present invention

FIGS. 16A and 16B are simplified schematic illustrations describing a packaging configuration that may be used for collapsing a self-adhesive mesh in accordance with some embodiments of the present invention;

FIGS. 17A, 17B and 17C are simplified schematic illustrations describing an alternate packaging configuration that may be used for collapsing a self-adhesive mesh in accordance with some embodiments of the present invention;

FIGS. 18A, 18B and 18C are simplified schematic illustrations of an exemplary removable sheath and underlying packaged mesh in accordance with some embodiments of the present invention;

FIGS. 19A and 19B are simplified schematic illustrations describing an exemplary method for vacuum packing the prepackaged mesh prior to delivery in accordance with some embodiments of the present invention;

FIGS. 20A and 20B are simplified schematic illustrations describing an exemplary method for compressing the prepackaged mesh prior to delivery in accordance with some embodiments of the present invention;

FIG. 21A is an illustration of an exemplary operative channel in accordance with some embodiments of the present invention;

FIG. 21B is an illustration of an adaptor which includes the funnel opening, the prongs, and a trocar connection element which are depicted in FIG. 21A and designed to be inserted into an existing trocar in accordance with some embodiments of the present invention;

FIG. 21C is an illustration of an exemplary guide rod with self-extending elements, having a plurality of valves for maintaining abdominal pressure in accordance with some embodiments of the present invention;

FIG. 22 is an illustration of an exemplary hand guiding unit in accordance with some embodiments of the present invention;

FIG. 23 is a simplified flow chart describing an exemplary method for delivering and positioning a mesh during a laparoscopic procedure in accordance with some embodiments of the present invention;

FIGS. 24A, 24B, 24C and 24D are simplified schematic illustrations describing an exemplary method for delivering and positioning a mesh during a trans-abdominal umbilical hernia operation in accordance with some embodiments of the present invention; and

FIGS. 25A, 25B, 25C and 25D are simplified schematic illustrations describing an exemplary method for delivering and positioning a mesh as part of a natural orifice trans-luminal endoscopy procedure in accordance with some embodiments of the present invention;

FIG. 26 is simplified schematic illustration of an exemplary guide rod with self-extending elements as depicted in FIG. 12A, with supporting threads in accordance with some embodiments of the present invention;

FIGS. 27A-27C are simplified schematic illustrations of a displacement sheet in accordance with some embodiments of the present invention;

FIG. 28 is simplified schematic illustration of an exemplary guide rod with self-extending elements which spread a graft in accordance with some embodiments of the present invention; and

FIG. 29 is simplified schematic illustration of an exemplary guide rod 50 with self-extending elements 615 which spread an exemplary pouch.

### DESCRIPTION OF SPECIFIC EMBODIMENTS OF THE INVENTION

The present invention relates to delivery and positioning of a surgical patch or the like and more particularly, but not exclusively, surgical mesh delivery and positioning for minimally invasive hernioplasty procedures.

An aspect of some embodiments of the present invention provides for a modular delivery and positioning system including a hand guiding unit, an operating channel and a prepackaged mesh unit that work together to deliver and position the mesh while each of the units can be separately adapted to specific surgical applications and meshes. According to some embodiments of the present invention, selection of the different components of the system, e.g. hand guiding unit, operating channel and prepackaged mesh unit can be made on site so that the system is adapted to a particular surgical procedure and/or for particular working parameters. For example, for a selected prepackaged mesh unit can be used with a plurality of different length hand guide units and sized operating channels to accommodate for different working conditions during a surgical procedure and for different applications.

An aspect of some embodiments of the present invention provides for a modular prepackaged mesh unit that is used to package different size and types of meshes for different types of applications. According to some embodiments of the present invention, different size and type meshes may be accommodated in the prepackaged mesh unit and delivered and positioned with a same hand guiding unit and operating channel. Exemplary meshes may include different size rectangular, oval and horseshoe shaped meshes. Exemplary applications may include inguinal hernia, femoral hernia, umbilical hernia, incisional hernia, and diaphragmatic or hiatal hernia performed by laparoscopic, endoscopic and/or trans-abdominal procedures. According to some embodiments of the present invention, the prepackaged mesh unit (which includes the mesh) is delivered through the operating channel and positioned in a desired orientation and position with the hand guiding unit. In some exemplary embodiments, the mesh is prepackaged directly on the hand guide unit and a separate hand guide unit is not required.

The Prepackaged Mesh Unit

According to some embodiments of the present invention, the prepackaged mesh unit includes a mesh, one or more self-extending elements (and/or spreading elements), and a guide rod (and/or leading rod). According to some embodiments of the present invention, each of the elements of the prepackaged mesh unit and the unit itself can be adapted to a specific application and mesh while still maintaining workability with the other units of the system. According to some embodiments of the present invention, the number and type of self extending elements is adapted to the type of mesh, the size of the mesh and the application for which the mesh is used. Typically, the one or more self-extending elements are fixedly attached to one end of the guide rod. Typically, during packaging, the end of the guide rod that is connected to the self-extending elements is generally centrally positioned on the mesh and the mesh is collapsed, e.g. rolled and/or folded together with the self-extending element(s) spread over the mesh. According to some embodiments of the present invention, centrally positioning the tip of the guide rod with the hernia centers the mesh with the herniated region. Centrally positioning the guide rod with the hernia improves control and versatility in positioning of the mesh with the guide rod. Optionally, the guide rod can assume a plurality of angles with respect to the mesh so that the guide rod can access the herniated area from a plurality of angels. Optionally, the guide rod additionally includes a flexible joint along its length between the two ends of the guide rod.

According to some embodiments of the present invention, the method and shaped used for collapsing and expanding the mesh is adapted to a specific application and/or mesh. In some exemplary embodiments, an umbrella fold is used to collapse a mesh around the guide rod and the self-extending elements function as stretchers to expand the umbrella fold. In some exemplary embodiments, the umbrella fold is specifically applied to oval shaped meshes (including circular meshes) and used in exemplary applications such as umbilical hernias and incisional hernias. In some exemplary embodiments, vertical and horizontal double cylinder scroll fold is applied to rectangular and horseshoe surgical mesh shapes. Optionally, a diagonal double cylinder scroll fold is applied to rectangular meshes. In the diagonal double cylinder scroll fold, rolling of the mesh is initiated from two opposing corners of the mesh

In some exemplary embodiments, the mesh is an adhesive mesh, e.g. a mesh including an adhesive layer over one surface of the mesh. Optionally, when delivering an adhesive mesh, a protective cover that does not adhere to the adhesive layer is positioned over the mesh prior to collapsing the mesh, e.g. rolling the mesh into the package. Typically the protective cover is fixedly attached to the guide rod and removed from the body cavity together with removal of the guide rod and self-releasing mechanism.

In some hernia applications, e.g. umbilical hernia applications, the supporting wall for the mesh is on a surface of the mesh including the self-extending elements. In some exemplary embodiments, one or more bands are connected to the mesh structure and the self-extending elements are fed through the bands during packaging. In some exemplary embodiments, the bands are used to hold the self-extending element to the mesh while expanding the mesh, e.g. from an umbrella fold. Optionally, the self-extending elements are adapted to slip out of the bands after fixation of the mesh as the guide rod is pulled away from the mesh, e.g. out of the body cavity.

According to some embodiments of the present invention, the size, shape and number of self-extending elements are adapted to the mesh used, the collapsing method and the region in which the mesh is to be positioned. Typically, the self-extending elements are elongated elements that are flexible and elastic. Typically, the elastic property of the self-extending elements stores energy while collapsed in the packaging.

Typically, the energy required to spreading the mesh is provided by the collapsed self-extending elements. Optionally, the self release elements are constructed from a plurality, e.g. bundle of elastic wire, e.g. 0.4-0.5 mm nitinol wire. In some exemplary embodiments, the self-extending elements are webbed with flexible bands, wires, and/or strings. The present inventor has found that webbing the self-extending elements with string like structure can provide for increasing the rigidity of the elements during expansion, e.g. by limiting the range of motion of the self-extending elements with respect to each other. Furthermore, the present inventor has found that the webbed structure can further assist in expanding the mesh by flattening the mesh as the string structure is taut during expansion. The present inventor has found that the added rigidity and improved functionality of the webbed string structure can be provided without significantly increasing the volume of the prepackaged mesh unit. Typically, the length of the string is selected so that it is taut at the fully extended radial position of self-extending elements.

In some exemplary embodiments, the self-extending elements are locked with one or more locking mechanism to maintain the self-extending elements in a specified configuration until actively released. In some exemplary embodiments, the prepackaged mesh unit additionally includes one or more tubes extending along the guide rod and onto the mesh area through which biological glue may be introduced for fixating the mesh once delivered and/or positioned.

According to some embodiments of the present invention the guide rod is a rigid rod with a tapered end adapted to be easily and repeatedly latched and released from the hand guiding unit. In some exemplary embodiments, the guide rod includes a notch and/or groove near the tapered end for clasping the guide rod onto the hand guiding unit. In some exemplary embodiments, the guide rod includes one or more prongs along the circumference of the rod adapted to fit between the prongs in the hand guiding unit and thereby prevent rotation of the guide rod when latched onto the hand guiding unit.

According to some embodiments of the present invention, the end of the guide rod that is adapted for attaching to the hand guide unit is standard for different meshes and different applications so that different meshes can be delivered, positioned and spread with a same hand guide unit. According to some embodiments of the present invention, the length of the guide rod is adapted to the length of the mesh when in a collapsed form. In some exemplary embodiments the rod includes a friction grip area around its circumference to improve its ability to be grasped and manipulated by graspers, e.g. for positioning of the mesh once delivered.

According to some embodiments of the present invention, the guide rod includes a connecting element for connecting the self-extending elements to one end of the guide rod. Optionally, the connecting element includes a collapsing mechanism for selectively collapsing the self-extending elements without the mesh after mesh deployment and prior to removing the self-extending elements from the body cavity. According to some embodiments of the present invention, the connection provides flexibility so that a range angles between the longitudinal axis of the guide rod and the self-extending elements can be achieved. This allows manipulation of the mesh from different angles. In some exemplary embodiments, a circumferential ring clamps the self-extending elements to the guide rod.

According to some embodiments of the present invention, for prepackaged mesh units where the mesh is rolled, the self-extending elements are introduced into the mesh when the mesh is partially rolled, e.g. the mesh partially rolled into a double cylinder scroll. In such embodiments, the self-extending elements are adapted to expand over the area of the mesh as it is released from its packaged state. In some exemplary embodiments, introducing the self-extending elements into the mesh once it is partially rolled, e.g. mostly rolled simplifies the packaging procedure.

According to some embodiments of the present invention, the prepackaged mesh unit is stored with the mesh in a collapsed state and unit is subsequently further collapsed, e.g. compressed prior to delivery. The present inventor has found that compressing the mesh prior to delivery can provide for reducing the volume of the mesh unit while avoiding possible damage to the mesh that may be caused by prolonged compression of the mesh during storage. In some exemplary embodiments, the prepackaged mesh unit is inserted into a sheath. Optionally, the sheath is vacuum packed prior to delivery. Optionally the sheath is elastic and prepackaged mesh unit is inserted into an elastic sheath prior to delivery to compress the prepackaged mesh unit. Optionally, the mesh is compressed during delivery, through a channel used to deliver the mesh into the body cavity.

The Operating Channel

According to some embodiments of the present invention the operating channel is a cylindrical channel that is adapted to the size of the prepackaged mesh unit to the operating channel, when required. Typically, the operating channel includes one or more extensions that can be fitted into each other (or connected to each other) to adapt the length for the operating channel. In some exemplary embodiments, the attachments can be removed after mesh delivery to provide improved control and agility during mesh positioning. According to some embodiments of the present invention, a diameter of the operating channel is selected to match the diameter of the prepackaged mesh unit and/or a trocar diameter. In some exemplary embodiments, the operating channel is used in place of the trocar, and its geometry is suitable for insertion into a port site. Optionally, the operating channel includes ridges along its circumference that are perpendicular to the longitudinal axis of the channel and used to resist slippage through a port hole. Optionally, an opening of the operating channel on one end is slanted so that it opens the port site as it is inserted through. Optionally the channel includes a handle positioned around the circumference of the channel for gripping and manipulating the channel.

The Hand Guiding Unit

According to some embodiments of the present invention, the hand guiding unit includes a cylindrical handle that allows manipulating the unit by hand from a plurality of hand grip directions, a handle rod connected to the handle on a first end and adapted to receive a guide rod on an opposite end, a locking mechanism for alternatively locking and releasing the guide rod from the handle rod, and a trigger mechanism for ejecting the guide rod from the handle rod when released. Optionally, the hand guiding unit includes a marker that indicates to the user that the guide rod has been released. Typically, the locking mechanism includes a locking knob and the triggering mechanism includes a triggering knob to control the locking and ejection of the guide rod. In some exemplary embodiments, the locking knob and the triggering knob is positioned on the handle rod proximal to the handle and/or on the handle. Optionally, the locking mechanism includes a latch positioned on the handle rod at a distal end from the handle and adapted to clasp the guide rod once inserted into the handle rod. Optionally, release of the latch is controlled by the locking knob. Optionally, the triggering mechanism includes a rod movably fed through the handle rod. Optionally, the trigger knob is adapted to extend the inner rod to push out the guide rod received by the hand guiding unit. Optionally, the length and of the handle rod is selected based on a depth of the hernia site while the diameter of the handle rod is compatible with the diameter of the guide rod. Optionally, the handle includes one or more release buttons for selectively releasing one or more of the self-extending elements from a locked state.

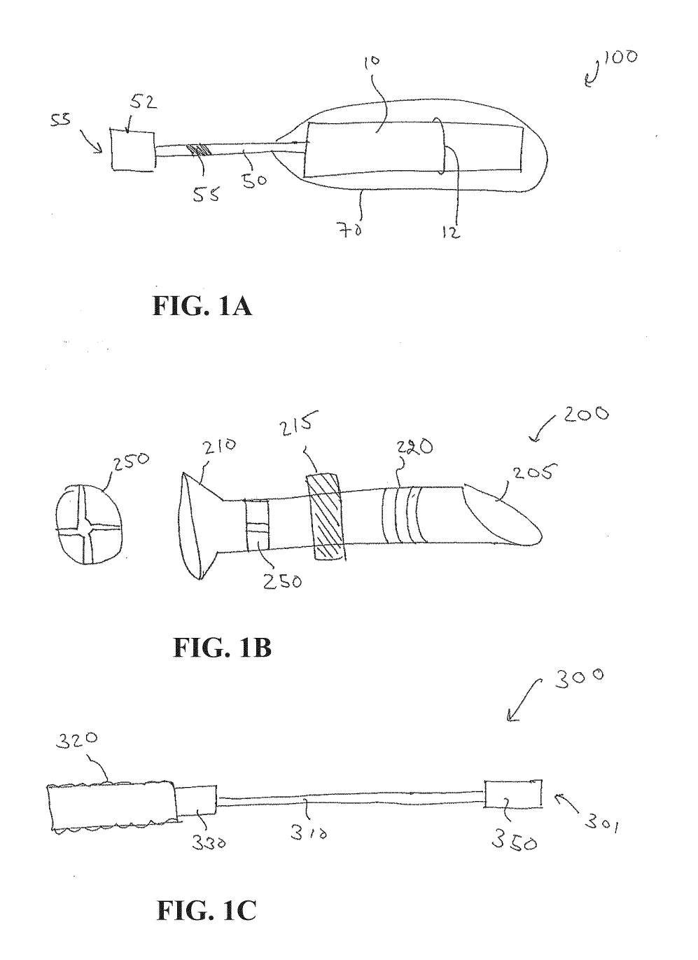

Reference is now made to FIGS. 1A, 1B and 1C showing simplified schematic illustrations of three basic units making up the modular surgical mesh delivery and positioning system in accordance with some embodiments of the present invention. According to some embodiments of the present invention, the basic units of the surgical mesh delivery and positioning system include a prepackaged mesh unit 100 including a surgical mesh 10, and operative channel 200 through which prepackaged mesh unit 100 is delivered and a hand guiding unit 300 adapted to engage prepackaged mesh unit 100 and to control delivery and positioning of mesh 10.

According to some embodiments of the present invention, mesh 10 is collapsed with one or more self-extending elements attached to a guide rod 50. Typically a clasp, e.g. a spring loaded clasp or band 12 holds mesh 10 in a collapsed state within prepackaged mesh unit 100. Optionally a protective sheath 70 is wrapped around the mesh. Optionally, sheath 70 is constructed from a transparent material. In some exemplary embodiments, sheath 70 protects the mesh from contact with the skin and is removed during delivery. In some exemplary embodiments, and as will be described in further detail herein sheath 70 is further used to compress mesh 10 prior to delivery through operative channel 200. According to some embodiments of the present invention, the diameter and/or length of prepackaged mesh unit 100 is adapted to a size and shape of mesh 10 as well as to the procedure being performed as is explained in further detailed herein.

According to some embodiments of the present invention, guide rod 50 includes a mechanism and/or structure 52 on or near its exposed end 55 adapted for engaging and locking into and/or onto hand guide unit 300. According to some embodiments of the present invention, mechanism 52 is adapted for repeated engagement and disengagement with hand guide unit 300 on demand during delivery and positioning of a mesh. Optionally, guide rod 50 includes a friction grip area 54 so that guide rod 50 can be easily griped and manipulated by surgical tools, e.g. claspers. Alternatively, guide rod 50 and handle guide unit 300 is a single unit and cannot be disengaged.

Referring now to FIG. 1B, according to some embodiments of the present invention, operative channel 200 has a length and inner diameter that corresponds to a length and diameter of prepackaged mesh unit 100 and/or mesh 10. In some exemplary embodiments, an inner diameter of operative channel 200 is smaller than a diameter of prepackaged mesh unit 100 and insertion of unit 100 into operative channel 200 provides for compressing unit 100. Optionally, operative channel 200 includes a converging opening 210 adapted to receive prepackaged mesh unit 100.

According to some embodiments of the present invention, operative channel 200 is fitted with a removable plug 250 operable to maintain abdominal and/or cavity pressure during mesh delivery and positioning. Optionally, plug 250 includes a plurality of flaps 251 between which guide rod 50 can be fitted. Optionally, plug 250 advances together with guide rod 50 to deliver mesh 10. Typically plug 250 is inserted subsequent to insertion of prepackaged mesh unit 100 and/or may be positioned on guide rod 50 prior to insertion into operative channel 200.

In some exemplary embodiments, operative channel 200 is adapted for insertion through a trocar. In some exemplary embodiments, operative channel 200 is additionally or alternatively adapted for insertion through a surgical port hole, e.g. upon removal of the trocar. Optionally, operative channel 200 includes rib markings 220 to resist slippage through a port site. In some exemplary embodiments, operative channel 200 includes a grip handle 215 for manipulating operative channel 200 and/or for preventing slippage for operative channel 200 through a surgical port site. In some exemplary embodiments, operative channel 200 is adapted for insertion instead of a trocar.

Optionally, end 205 of operative channel 200 is slanted so that as operative channel 200 is inserted through a port hole, the hole opens up. In some exemplary embodiments, operative channel 200 is not required and the prepackaged mesh unit is delivered through a trocar or an endoscope, e.g. during normal orifice transendoscopic surgery (NOTES).

Typically, operative channel 200 is used to deliver prepackaged mesh unit 100. According to some embodiments of the present invention, operative channel 200 is additional operable as an operative channel through which hand guide unit 300 or other surgical tools are introduced to an operating site. According to some embodiments of the present invention, operative channel 200 is constructed from transparent material and prepackaged mesh unit 100 can be viewed via the channel wall.

Referring now to FIG. 1C, according to some embodiments of the present invention, hand guide unit 300 includes a hand grip 320 and a handle rod 310 extending from hand grip 320. In some exemplary embodiments, handle rod 310 includes a lock mechanism 350 on an end 301 distal to hand grip 330 operable to latch on and release guide rod 50. In some exemplary embodiments, handle rod 310 includes hand controls 330 proximal to hand grip 320 for controlling locking and releasing of guide rod 50. Optionally, hand controls 330 provide for selectively releasing one or more of the self-extending elements and/or locking elements in prepackaged unit 100. Typically, remote release of the self-extending elements with hand controls 330 are provided when the hand guide 300 and guide rod 50 are integrated in one unit, e.g. the self extending elements and mesh are directly connected to the end of the hand guide.

The Prepackaged Mesh Unit

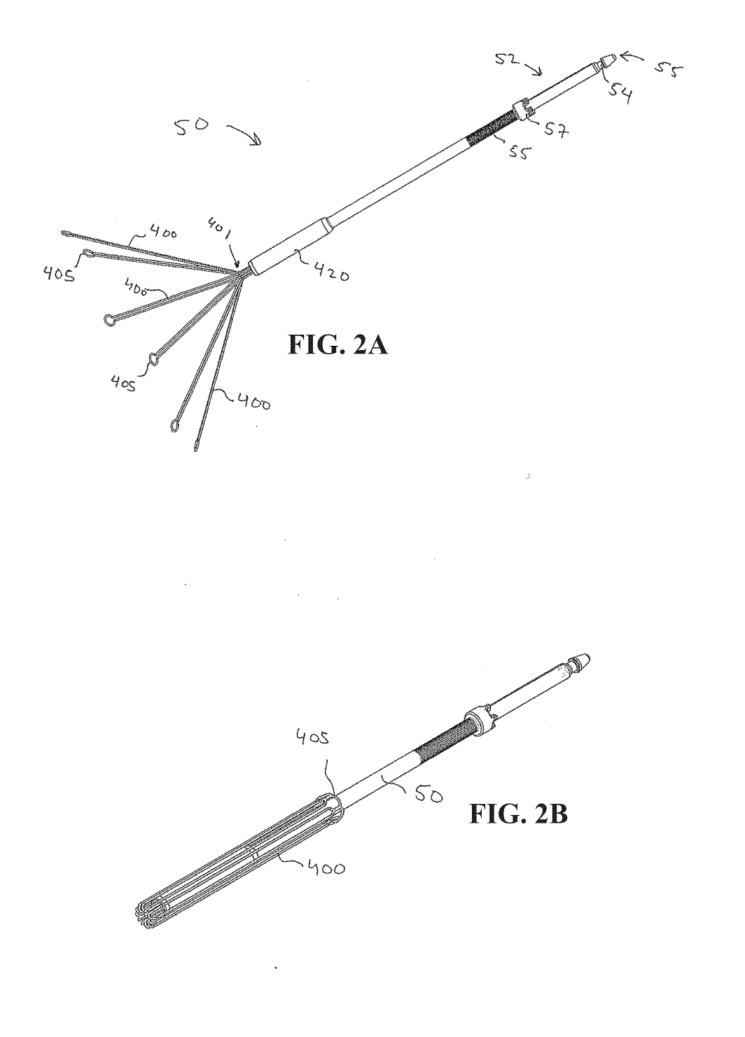

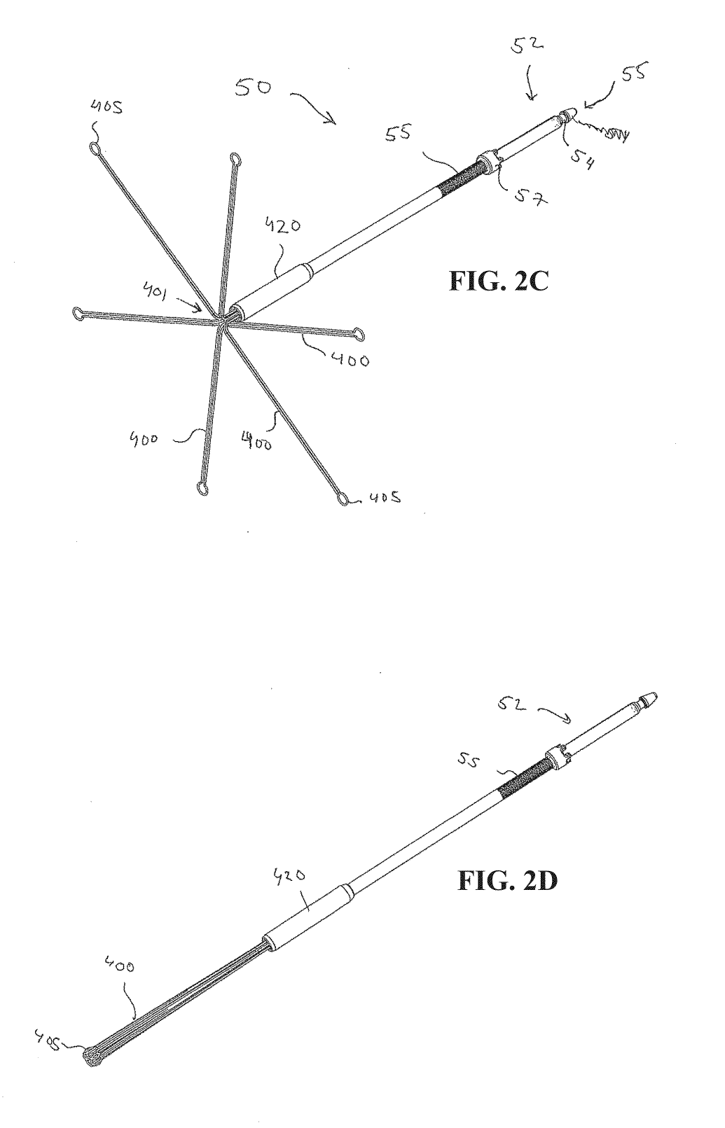

Reference is now made to FIGS. 2A, 2B, 2C and 2C showing illustrations of an exemplary guide rod including exemplary self-extending elements in four different positions in accordance with some embodiments of the present invention. According to some embodiments of the present invention, one or more flexible self-extending elements 400 are connected to guide rod 50. Optionally, self-extending elements 400 are only flexible around bending point or area 401.

In some exemplary embodiments, self-extending elements 400 are in the form of a wire forming a loop 405 at one end. Loops 405 may provide an extended surface area for holding down mesh 10 during placement. Optionally, loops 405 include ribs or stripes to increase the frictional hold on mesh 10. Optionally, loop 405 can be replaced by a pin that penetrates through the mesh and thereby is removably attached to the mesh. Alternatively, self-extending elements 400 may be constructed from stripes or bands, e.g. super-elastic bands. The stripes, bands or wire can optionally be constructed from plastic polymer, metals and/or alloys such as Nickel Titanium or other shape memory alloys.

According to some embodiments of the present invention, connecting element 420 fixedly connects the self-extending elements 400 to guide rod 50. In some exemplary embodiments, connecting element 420 is flexible or provides for flexible connection so that guide rod 50 can manipulate mesh 10 with self-extending elements 420 from a range of angles.

In some exemplary embodiments, exposed end 55 is pointed so that it can be easily inserted into hand guide 300 (FIG. 1C). In some exemplary embodiments structure 52 adapted for engagement with hand guide unit 300 includes a groove54 that can be used to lock guide rod 50 on hand guide 300. Optionally, structure 52 additionally includes one or more spokes 57 adapted to engage hand guide unit 300 to prevent rotation between hand guide unit 300 and guide rod 100.

In some exemplary embodiments, the positioning of self-extending elements, shown in FIG. 2A, represents an unloaded position of the self extending elements 400. According to some embodiments of the present invention, bending extending elements 400, e.g. at a bending point 401, provides for loading a spring force on self-extending elements 400 so that they self-extend when released from the loaded position.

Referring now to FIG. 2B, self-extending elements 400 are positioned in a loaded position while packaged in prepackaged mesh unit 100 in accordance with some embodiments of the present invention. In some exemplary embodiments, self-extending elements are bent back toward guide rod 50 while packaged in prepackaged mesh unit 100. Optionally, self-extending elements 400 have different lengths so that they do not form a bulge when brought together in the prepackaged mesh unit. In some exemplary embodiments, mesh 10 is wrapped over self-extending unit in the form of a folded umbrella.

FIG. 2C shows self-extending elements 400 in fully radially extended positions. In some exemplary embodiments, while the self-extended elements are fully extended, the mesh is unraveled and/or expanded and can be positioned by panning guide rod 50. Typically, fully radially extended position of self-extended elements is achieved by pressing the self-extended elements against a wall including the herniated area. Optionally, at a first stage half of self-extending elements 400 are released and centered over the herniated area. The present inventor has found that unraveling one half of the mesh at a time, provides improved visibility while centering the mesh over the herniated area since a portion of the hernia can still be viewed on the unraveled side. Once centered, in the second half of the self-extending elements are released. Alternatively, the entire mesh is released and spread simultaneously.

FIG. 2D shows self-extending elements 400 in fully longitudinally extended positions. Optionally, this longitudinal extended position of the self-extending elements is the natural and/or unloaded position of the self-extending elements. In some exemplary embodiments, subsequent to positioning of mesh 10, guide rod 50 is removed from the body cavity through channel 200 urging the self-extending elements 400 in fully longitudinally extended positions as they are pulled through channel 200.

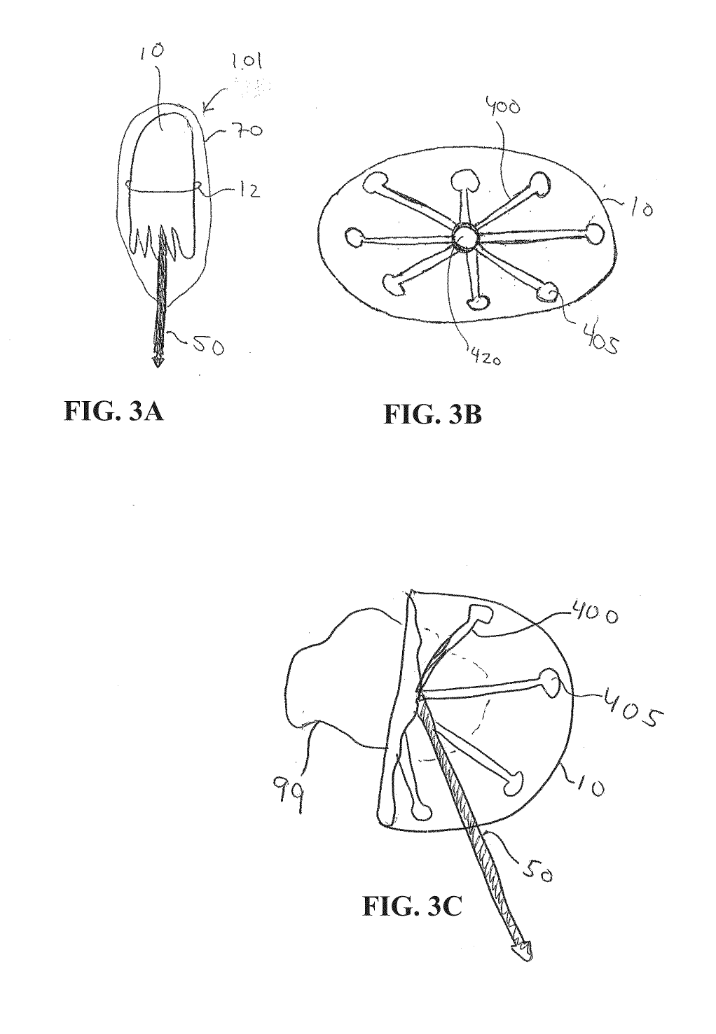

Reference is now made to FIGS. 3A, 3B and 3C showing simplified schematic illustrations describing an umbrella packaging method that may be used for collapsing a mesh in the prepackaged mesh unit in accordance with some embodiments of the present invention. According to some embodiments of the present invention, prepackaged mesh unit 101 includes a mesh 10 and guide rod 50 packaged using an umbrella packaging method. According to some embodiments of the present invention, to package mesh 10, self-extending elements 400 are spread over mesh 10 (FIG. 3B) and then bent back toward guide rod 50 as shown in FIG. 2B to form an umbrella fold as illustrated in FIG. 3A. Optionally, removing sheath 70 and releasing band 12 initiates opening of mesh 10 in response to extension of loaded self-extending elements 400. Optionally, the angle of extension of self-extending elements 400 is adapted to geometry of a surface of the herniated area. Typically, oval (including round) meshes are used in the umbrella fold. Optionally, other shaped meshes, e.g. rectangular (including square) and horseshoe shaped meshes may be used.

Referring now to FIG. 3C, in some exemplary embodiments, during a first stage of deployment, half of the self-extending elements are released and the mesh 10 is centered over a herniated area 99 with guide rod 50. The present inventor has found that releasing only half of the self-extending elements during this first stage of deployment provides for increased visibility that of the herniated area and aids in properly centering mesh 10. In some exemplary embodiments, once the mesh is centered, the rest of self-extending elements are released. Optionally, a releasing mechanism is used to selectively release half of the self-extending elements 400 during each stage as will be described in more detail herein below.

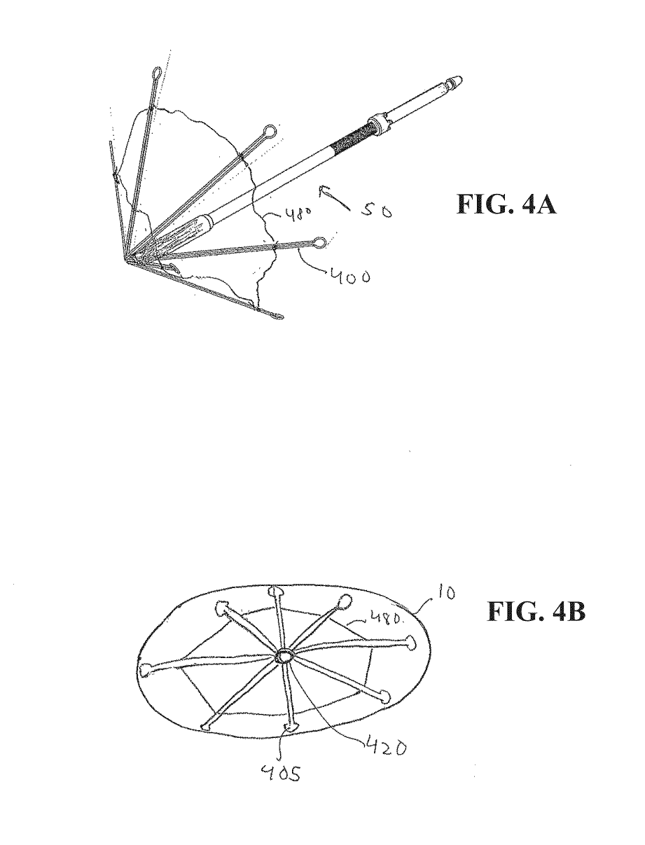

Reference is now made to FIGS. 4A and 4B showing simplified schematic illustrations of an exemplary guide rode including webbed self-extending elements for expanding an umbrella packaged mesh in accordance with some embodiments of the present invention. According to some embodiments of the present invention, a flexible string-like structure, e.g. a string or wire 480 is connected between the self-extending elements. In some exemplary embodiments, string 480 limits the movement of the self-extending element toward and away from each other, increases the rigidity of the self-extending elements and helps flatten mesh 10 in the area between the self-extending elements.

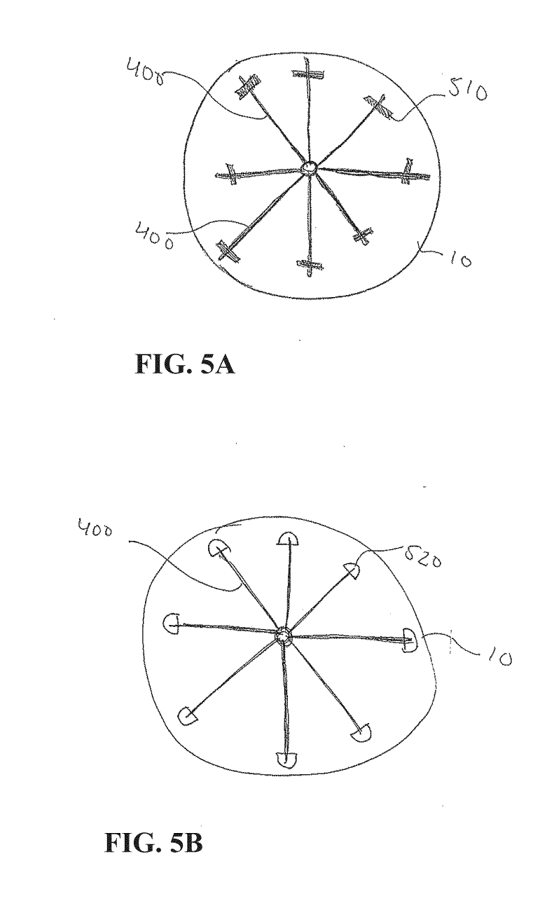

Reference is now made to FIGS. 5A and 5B showing simplified schematic illustrations of a mesh including exemplary restraining elements to hold the self-extending elements during self-expansion of the prepackaged mesh unit in accordance with some embodiments of the present invention. According to some embodiments of the present invention, one or more bands 510 and/or pockets 520 are secured to mesh 10 and self-extending elements are fitted into bands 510 and/or pockets 520 during packaging. According to some embodiments of the present invention, bands 510 and/or pockets 520 can help prevent mesh 10 from shooting off the self-extending elements upon release. Optionally, bands 510 and/or pockets 520 can be constructed from the same or similar material to grid 10. Optionally, bands 510 and/or pockets 520 are constructed from an elastic material. Applying bands 510 and/or pockets 520 on mesh 10 is particularly useful for a hernia repair where the herniated wall is opposite the surface including the self-extending elements, e.g. umbilical hernia procedure, so that there is no wall that can be used as a contra force to the self-extending elements.

According to some embodiments of the present invention, self-extending elements 400 are disengaged from bands 510 and/or pockets 520 after fixating the mesh by retracting guide rod 50 out of the body cavity. Optionally, the band is replaced by a staple. As guide rod 50 is retracted, self-extending elements 400 slip out of bands 510, staples and/or pockets 520 and revert to their unload position (FIG. 2A). Further retraction of guide rod 50 through operative channel 200 gathers self-extending elements together as shown in FIG. 2D so that guide rod 50 can be removed through the channel. In some exemplary embodiments, bands 510 and/or pockets 520 may be used for meshes packaged in a variety of folding methods, e.g. an umbrella fold and/or in a double cylinder scroll fold. Alternatively, ends 405 of self-extending elements 400 are fitted through the mesh, e.g. though a small hole introduced into the mesh to secure ends 405 to the mesh. Optionally, subsequent to mesh deployment and fixation, the ends 405 are pulled out of the mesh.

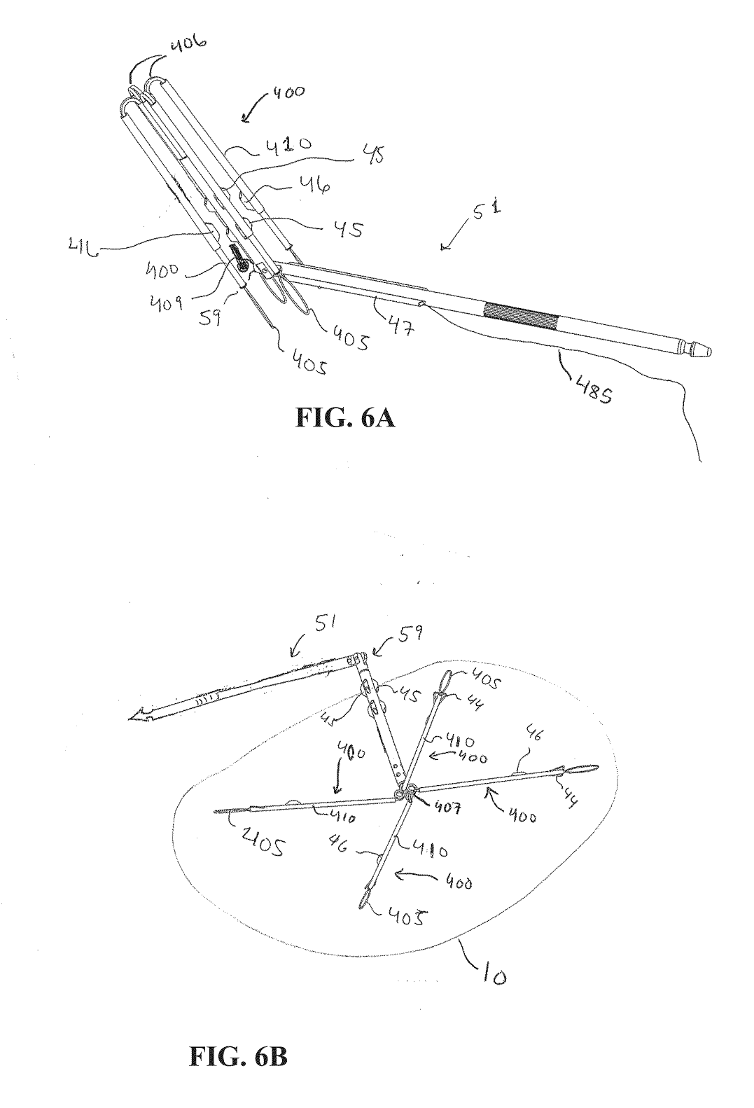

Reference is now made to FIGS. 6A and 6B showing illustrations of two additional exemplary guide rods including exemplary self-extending elements for deploying a mesh in an umbrella fold in accordance with some embodiments of the present invention. According to some embodiments of the present invention guide rod 51 includes a flexible joint 59 that can be bent to access a target area, e.g. a herniated area from different angles. In some exemplary embodiments self-extending elements 400 are locked in a collapsed state with one or more pins 409 fitted through channels 45 on guide rod 51 and channels 46 on self-extending elements 400 that align with each other while the self-extending elements are collapsed. Optionally, each pin 409 (shown as disengaged from channels 45 and 46) is connected to a string or wire 485 fitted through an additional guide channel 47 and released from channels 45 and 46 in response to pulling of wire 485. Optionally, a button or latch on a handle of hand guiding unit 300 (FIG. 1C) provides a pull force that releases one or more pins 409. Pin 409 can then be removed from the body cavity together with guide rid 501. Optionally, one or more pins 409 are removed with a surgical tool introduced into the body cavity through a surgical port. Typically, half the self-extending elements 400 are released, e.g. simultaneously released during a first stage of deployment and the other half is released at a second stage of deployment.

According to some embodiments of the present invention, self-extending elements 400 are constructed from bands elastic material 406 or a coil of elastic material 407 partially covered by a rigid covering 410. Optionally, covering 410 is a tube fitted over the elastic material and includes one or more channels 45 for selectively locking to prevent elements 400 from extending into their neutral configuration and releasing self-extending elements 400 to allow elements 400 to extend to their neutral configuration.

Referring now to FIG. 6A, in some exemplary embodiments, elastic material 406 included in each self-extending arm 400 is a plurality of elastic and/or shape memory wire. Optionally, elastic material 406 includes a bundle of 0.4-0.5 mm nickel titanium wire. Optionally, when released, the nickel titanium extends from its locked position to a neutral position at a 150 degree angle.

Referring now to FIG. 6B, in some exemplary embodiments, elastic material 407 includes a coil configuration that provides the elastic properties required to self extend self-extending elements 400. In some exemplary embodiments, elastic material 407 is constructed from stainless steel. Optionally, elastic material 407 is constructed from a shape memory alloy such as nickel titanium. Furthermore, in some exemplary embodiments, when self-extending element 400 includes a loop 405 or other end element adapted to be inserted through a mesh 10 and thereby be fixated on the mesh, self-extending element 400 additionally includes a stopper element 44 for limiting penetration of self-extending element 400 through mesh 10. Optionally, loop 405 can be replaced by a pin that penetrates through the mesh with stopper element 44 restricting the amount of penetration through the mesh. Typically stopper element 44 includes arms that extend outwardly from self-extending elements and thereby block penetration of the self-extending elements past the stopper element 44. Optionally, stopper elements 44 are integrated and/or positioned on rigid covering 410.

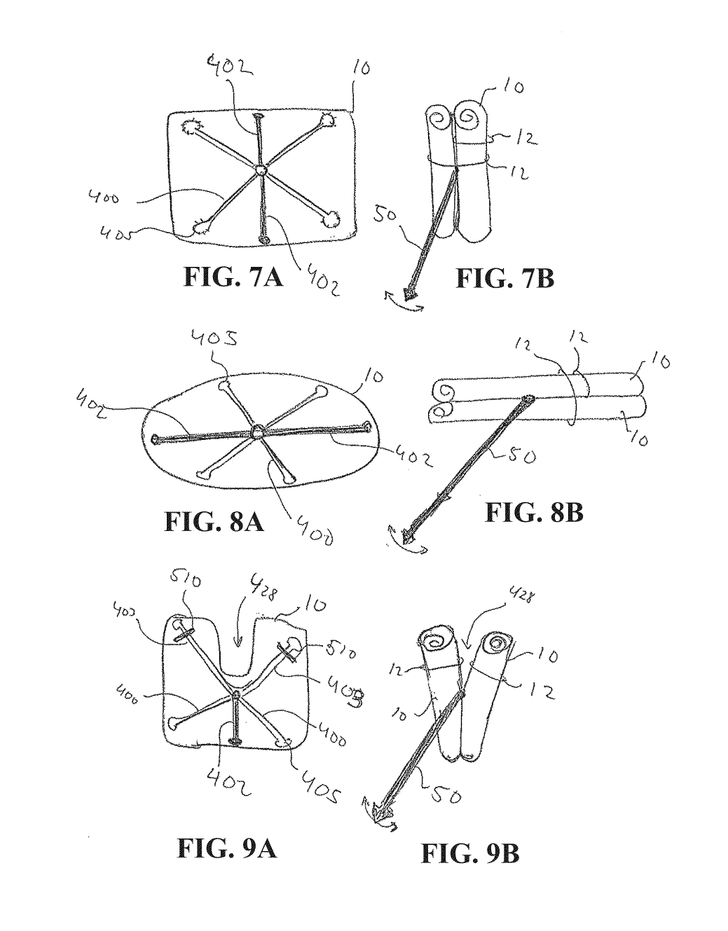

Reference is now made to FIGS. 7A and 7B showing simplified schematic illustration describing an exemplary vertical double cylinder scroll packaging method that may be used for collapsing a mesh in the prepackaged mesh unit in accordance with some embodiments of the present invention. According to some embodiments of the present invention, self-extending elements 400 include flexible elements that may be rolled together with mesh 10 in a double cylinder scroll. Optionally, guide rod 50 may include one or more elements 402 that are rigid and are vertically positioned along a central axis of mesh 10 defined by the line along which the two cylinders meet. In some exemplary embodiments, guide rod 50 is similar to guide rod 50 described in reference to FIGS. 2A-2D. According to some exemplary embodiments, guide rod 50 has a flexible connection between the guide rod and the self-extending elements 400 and elements 402. The flexible connection enables controlling positioning of mesh 10 from a variety of angles of guide rod 50. The double cylinder scroll folding method may be applied to rectangular, oval and horseshoe shaped meshes.

According to some embodiments of the present invention, double cylinder scroll is packaged with at least two fasteners 12 to enable releasing (unrolling) and fixating one cylinder at a time.

Reference is now made to FIGS. 8A and 8B showing simplified schematic illustration describing an exemplary horizontal double cylinder scroll packaging method that may be used for collapsing a mesh in the prepackaged mesh unit in accordance with some embodiments of the present invention. According to some embodiments of the present invention, horizontal double cylinder scroll packaging method is similar to the packaging method described in reference to FIGS. 7A and 7B. According to some embodiments of the present invention guide rod 50 includes one or more supporting elements 402 are horizontally positioned along a central axis of mesh 10 defined by the line along which the two cylinders meet.

Additional details regarding the vertical and horizontal double cylinder packaging system and method can be found in incorporated Publication WO2006/082587 and can be applied herein.

Reference is now made to FIGS. 9A and 9B showing simplified schematic illustrations describing an exemplary horseshoe double cylinder scroll packaging method that may be used for collapsing a mesh in the prepackaged mesh unit in accordance with some embodiments of the present invention. Horseshoe shaped meshes are typically applied to hiatal hernias where the mesh is placed behind the stomach and around the esophagus. According to some embodiments of the present invention, self-extending elements 403 are structured to maintain opening 428 when rolled in a double cylinder scroll so that the horseshoe mesh can be positioned around the esophagus, for example, while in the rolled position. Optionally, bands 510 are applied to secure self-extending elements 403 to horseshoe mesh and thereby maintain opening 428 when rolled.

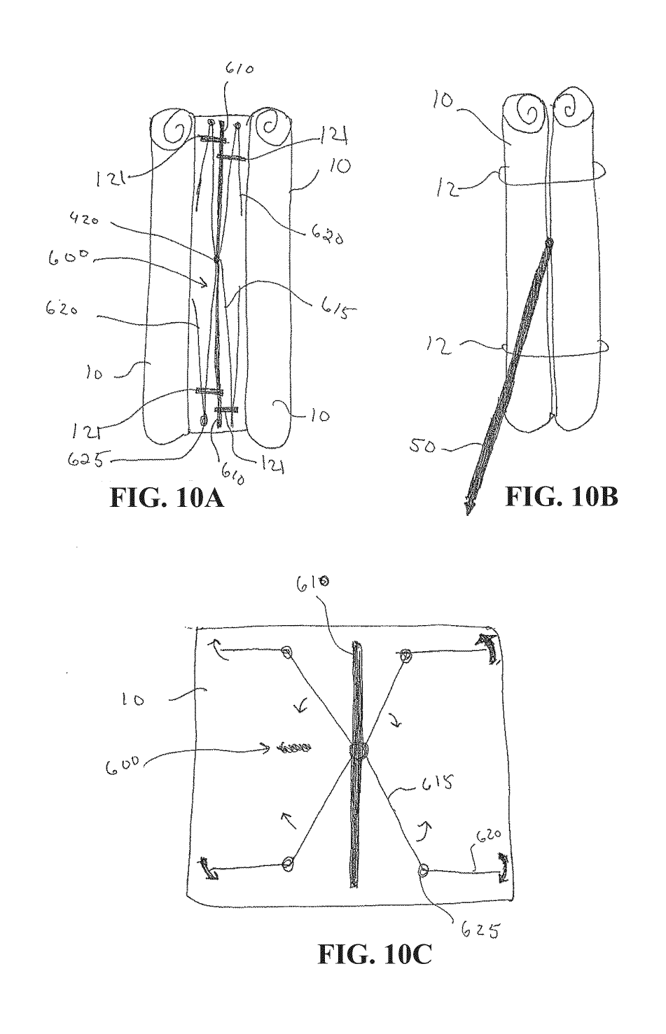

Reference is now made to FIGS. 10A, 10B, and 10C showing simplified schematic illustration of an exemplary self-extending unit adapted to be inserted into a partially rolled double cylinder scroll in accordance with some embodiments of the present invention. According to some embodiments of the present invention guide rod 50 is flexibly connected to a self-extending unit 600 and that be positioned within a partially rolled mesh 10 (FIG. 10A). The present inventor has found that inserting a self-extending unit into a partially rolled mesh avoids any difficulties that may be associated with rolling the mesh together with the self-extending elements. According to some embodiments of the present invention, self-extending unit includes bands 121 that maintain the self-extending unit in a packaged, e.g. collapsed position and provide for separately extending each cylinder of the mesh rolled in a double cylinder scroll. It is noted that in FIG. 10A, guide rod 50 is not shown so that the features of the self-extending unit 600 is not obstructed but it is clear that the guide rod connects to the self-extending unit at connecting area 420. Once the self-extending unit is inserted, mesh 10 is fully rolled and clasped with one or more clasps 12.

According to some embodiments of the present invention, self-extending unit 600 includes one or more supporting elements 610, optionally rigid, that extend along the central line of the double cylinder scroll, and self-extending elements 615 that extend away from supporting element 610 in an unloaded position. In some exemplary embodiments, self-extending unit 600 additional includes elements 620 connected to elements 615 by an elastic connection 625. According to some embodiments of the present invention, elements 620 extend away from element 615 in an unloaded position. According to some embodiments of the present invention, releasing self-extending unit 600 and allowing it to assume its unloaded position provides for unrolling (deploying) mesh 10.

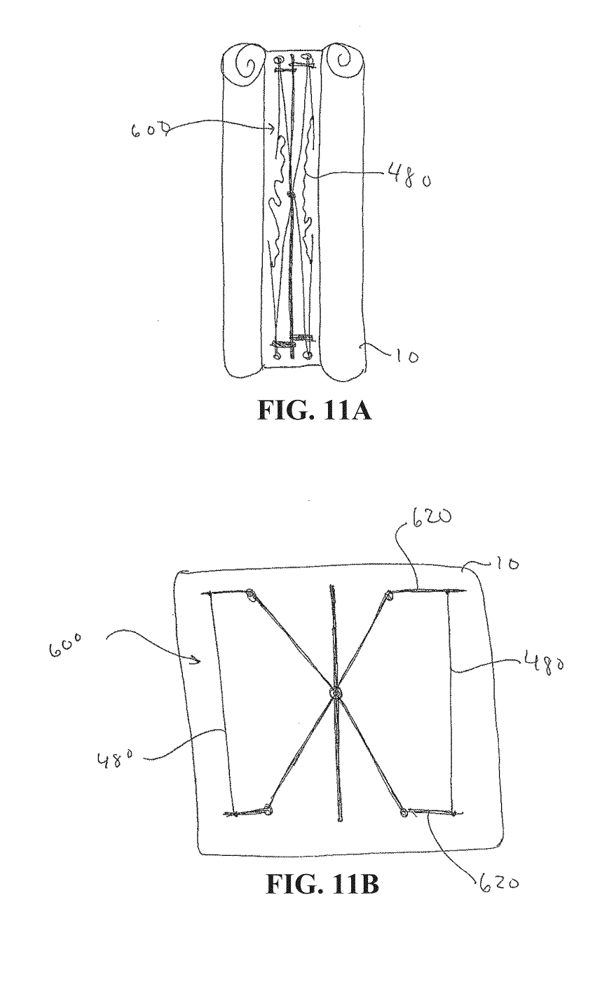

Reference is now made to FIGS. 11A and 11B showing simplified schematic illustration of an exemplary self-extending element including string structures adapted to be inserted into a partially rolled double cylinder scroll in accordance with some embodiments of the present invention. According to some embodiments of the present invention, a flexible string is connected between pairs of elements 620 to help spread and flatten mesh 10 as it unrolls. Typically, the length of the string is selected so that it is taut at the fully extended position of self-extending unit 600 (FIG. 11B).

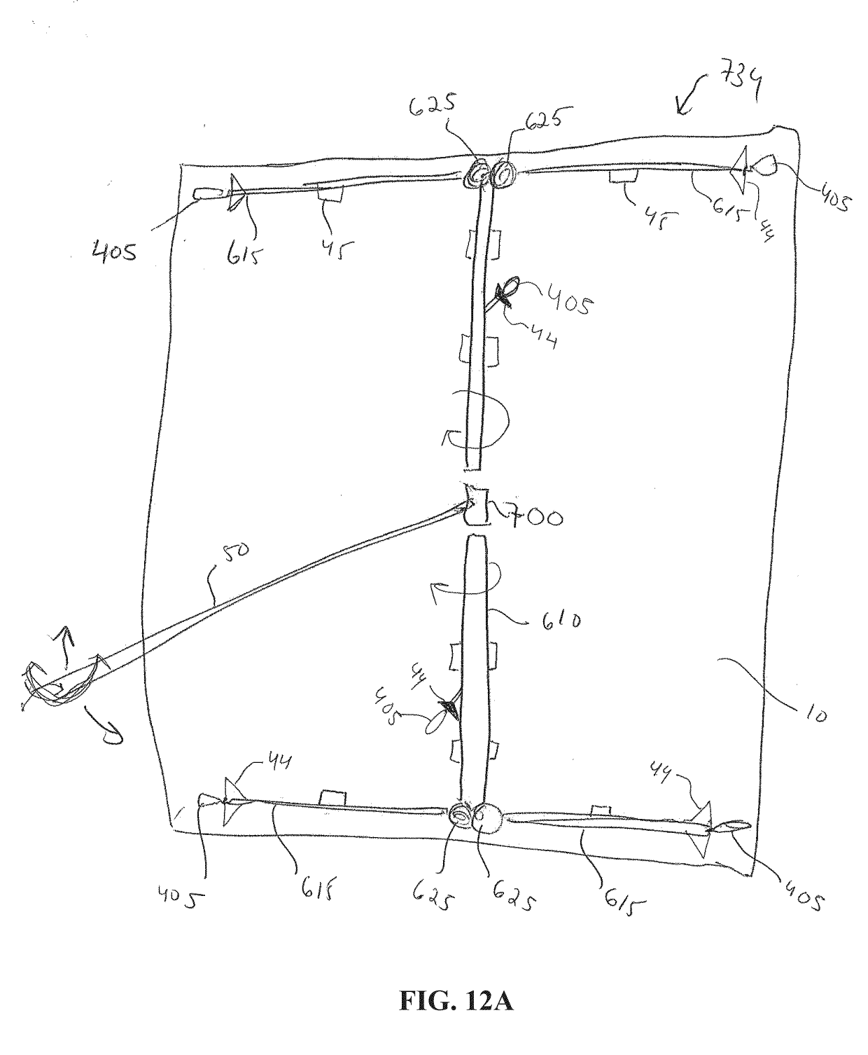

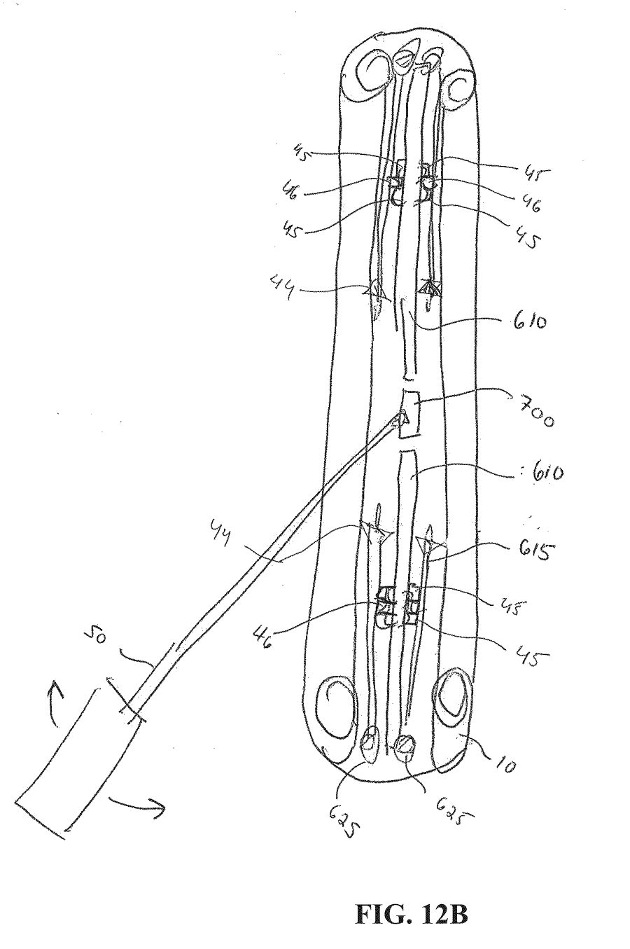

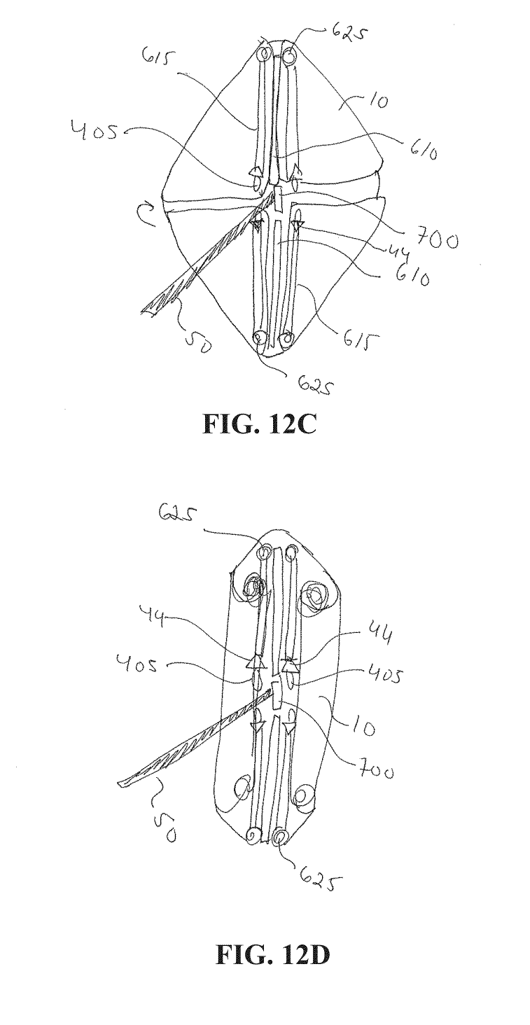

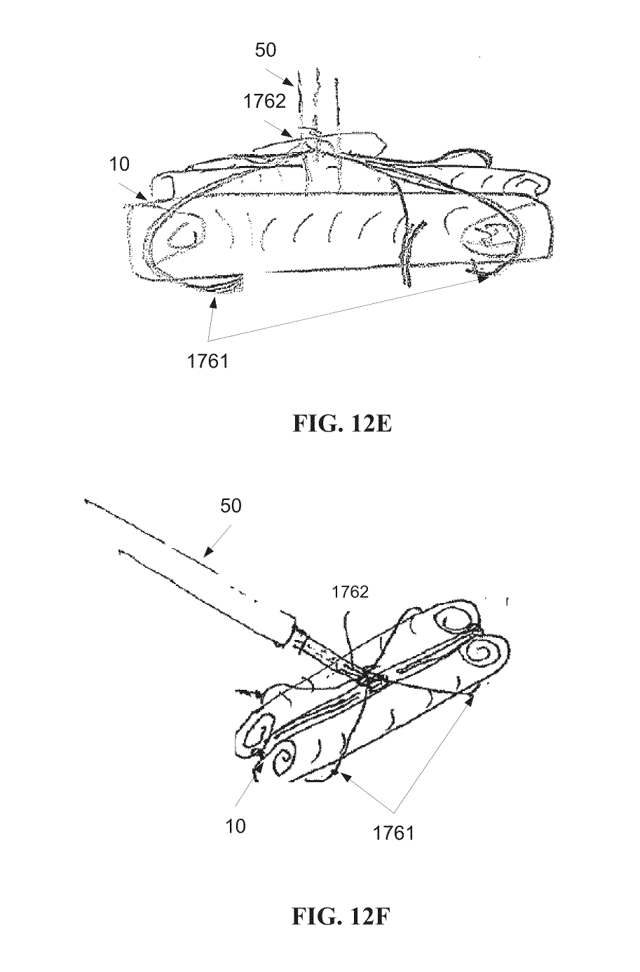

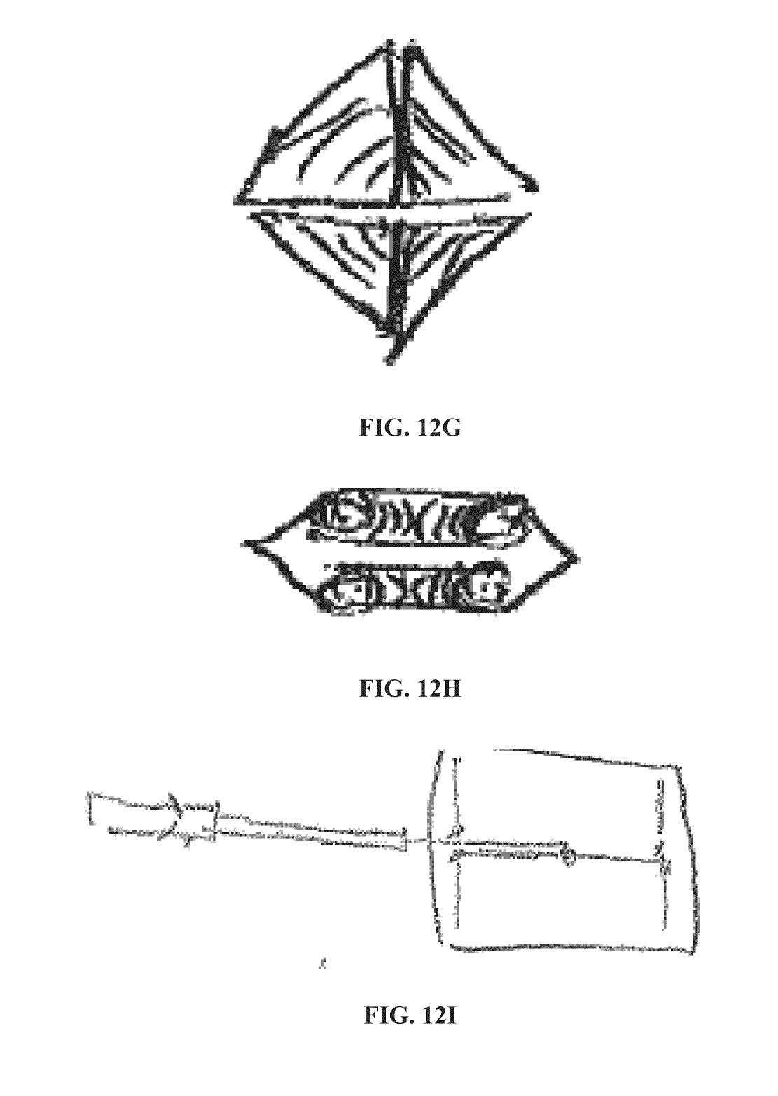

Reference is now made to FIGS. 12A and 12B showing simplified schematic illustrations of an exemplary guide rod with self-extending elements including locking mechanisms for controllably locking and releasing the self-extending elements in accordance with some embodiments of the present invention. According to some embodiments of the present invention, supporting elements 610 are connected to guide rod 50 through holder element 700. In some exemplary embodiments, guide rod 50 is connected to holder element 700 with rotatable connection, e.g. a hinge connection so that guide rod 50 can be collapsed with respect to supporting elements 610 during delivery and retraction and so that guide rod 50 can position mesh 10 to the herniated area from different angles. According to some embodiments of the present invention, holding element 700 maintains supporting elements 610 from either side of guide rod 50 extended at a 180 degree angle with respect to each other. Optionally, holding element 700 allows supporting elements 610 a certain degree of motion. Optionally, the holding element 700 allows rotating and tilting the supporting elements 610 in relation to the guide rod 50 so as to allow moving the guide rod 50 to any angle in a hemisphere formed above a plane that is parallel to the supporting elements 610. By facilitating such rotating and tilting, the mesh 10 may be angled to cover intrabody raptures in various location and angles, without having to change the angle of the guide rod 50 in relation to the body of the patient.

Optionally, supporting elements 610 can rotate with respect to holding element 700 over a range between 120-180 degrees. Optionally, this range of motion provides for spreading mesh 10 over a surface that is not flat. Optionally, central supporting elements 610 can be rotated about their longitudinal axis together with guide rod 50, e.g. in response to a corresponding rotational movement of guide rod 50.

According to some embodiments of the present invention, elements 615 include one or more channels or openings 45 and supporting element 610 includes one or more channels openings 46 through which locking pins, e.g. locking pin 409 (FIG. 6A) can be fitted. In response to removing the locking pins from openings 45 and 46, elements 615 extend from their folded position due to pre-stored energy stored in elastic connection 625 and spread mesh 10. Optionally, elements 615 are supporting elements. In some exemplary embodiments, ends and/or loops 405 are fed through the mesh, e.g. through a hole in the mesh to fixate, e.g. removably fixate the self-extending unit 734 to mesh 10. In some exemplary embodiments, a stopper element 44 limits the further penetration of elements 615 through the mesh. Optionally, ends and/or loops with stopper elements are also provided off of supporting elements 610.

Referring now to FIGS. 12C and 12D, in some exemplary embodiments, ends 405 are connected to mesh 10, e.g. corners of mesh 10 and the corners of mesh 10 are brought toward the center of the mesh while elements 615 are positioned in their collapsed state (FIG. 12C). Optionally, mesh 10 is further collapsed (FIG. 12D) by rolling in edges of mesh 10 distal from elements 610. In order to maintain the mesh 10 in a collapsed state, one or more threads, which are optionally connected to the tip of the guide rod 50, are used. The thread(s) are threaded via hole(s), optionally designated, in the mesh 10 and stretched around the collapsed mesh 10 to maintain the rolled shaped thereof. For example, FIGS. 12E and 12F depict the mesh 10 maintained by threads 1761 in a collapsed state.

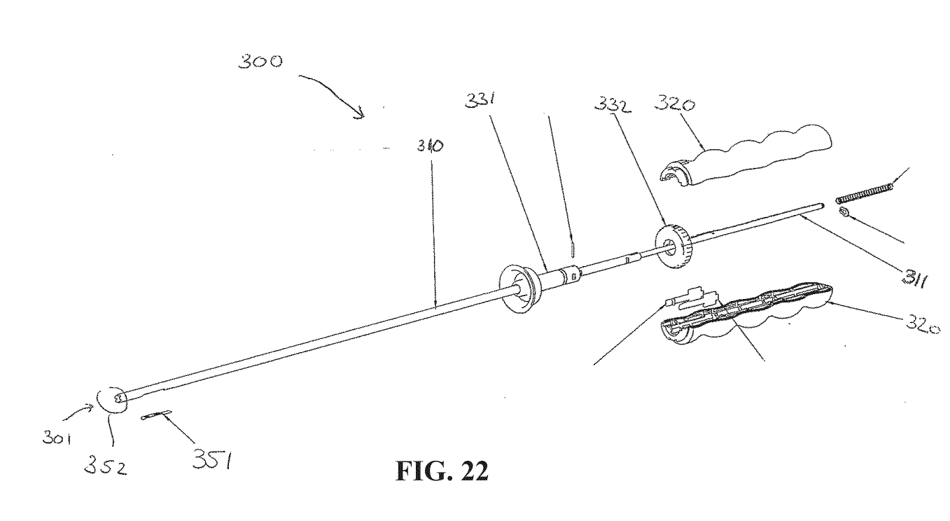

The stretched thread(s) are optionally connected to a releasing mechanism having one or more controlled pins, for example as depicted in 1762 of FIGS. 12E and F, at the tip of the guide rod 50. The releasing mechanism includes a mechanical control to adjust the location of the controlled pins so as to release the tension that is applied on the threads. Optionally, the mechanical control includes one or more mechanical triggers which are placed at a holding handle grip, for example as depicted in FIG. 22. Such triggers may be connected to one or more shafts that pull or push the controlled pins. Optionally, two sets, each of one or more threads, are separately connected to independent releasing mechanisms. In such a manner, a half of the mesh 10 may be deployed separately. By separately deploying each half, the physician gains more control on the deployment process. The deployment of one half of the mesh 10 instead of a full deployment of all the mesh 10 allows the physician to aim the deployment of the other half more accurately.

Optionally, in order to maintain the mesh in a folded state, for example as depicted in FIG. 12C, the self-extending elements 615, which are connected to the lateral sides of the mesh 10, are locked substantially in parallel to the supporting elements 610. The locking is performed by a locking mechanism that is optionally connected to the aforementioned releasing mechanism. In such a manner, slightly after the threads are released, the locking mechanism may allow the respective self-extending elements 615 to spread the mesh or a portion thereof. For example, the threads and the locking mechanism may be coordinated so that the self-extending elements 615 which are connected to a first half of the mesh 10 are released slightly after respective threads and the self-extending elements 615 which are connected to a second half of the mesh 10 are released slightly after respective other threads.

According to some embodiments of the present invention, a kit which includes the aforementioned exemplary self-extending unit is provided with a mesh, such a mesh 10, connected thereto. Optionally, the kit is provided with a mesh that is maintained in a collapsed state, for example as described above, for example as depicted in FIGS. 12D-12F. Optionally, the exemplary self-extending unit is provided with a mesh that is maintained in a semi collapsed state, for example as depicted in FIG. 12C. In such an embodiment, the mesh is, which is optionally folded from the four corners of the mesh, is rolled before the deployment begins, for example manually, by the physician. Optionally, locking mechanism automatically locks the self-extending elements 615 when the physician, or as a supporting mechanism, proximate them toward the supporting elements 610. When the mesh is provided in the semi collapsed state, the mesh is not exposed to the mechanical forces which are applied by the rolling before the deployment process begins.

Optionally, the exemplary self-extending unit is provided with a mesh that is maintained in a spread state, for example as depicted in FIG. 12I. In such an embodiment, the mesh is, folded from the four corners of the mesh, to the state depicted in FIG. 12C and then rolled before the deployment begins, for example to the collapsed state depicted in FIG. 12D, optionally manually, by the physician. When the mesh is provided in the spread state, the mesh is not exposed to the mechanical forces, which are applied by the folding and the rolling before the deployment process begins. For example, when the mesh is laminated with a bio compatible composite, such as collagen, avoiding the folding or rolling allows avoiding a shape memory effect. Optionally, the folding is performed by pulling threads, which are connected to the corners of the mesh, or by a designated manual device that aligns the elements 615 in parallel to the supporting elements 610.

It should be noted that if the mesh has a square shape the folding thereof may provide the shape depicted in FIG. 12G and rolled to the shape depicted in FIG. 12H.

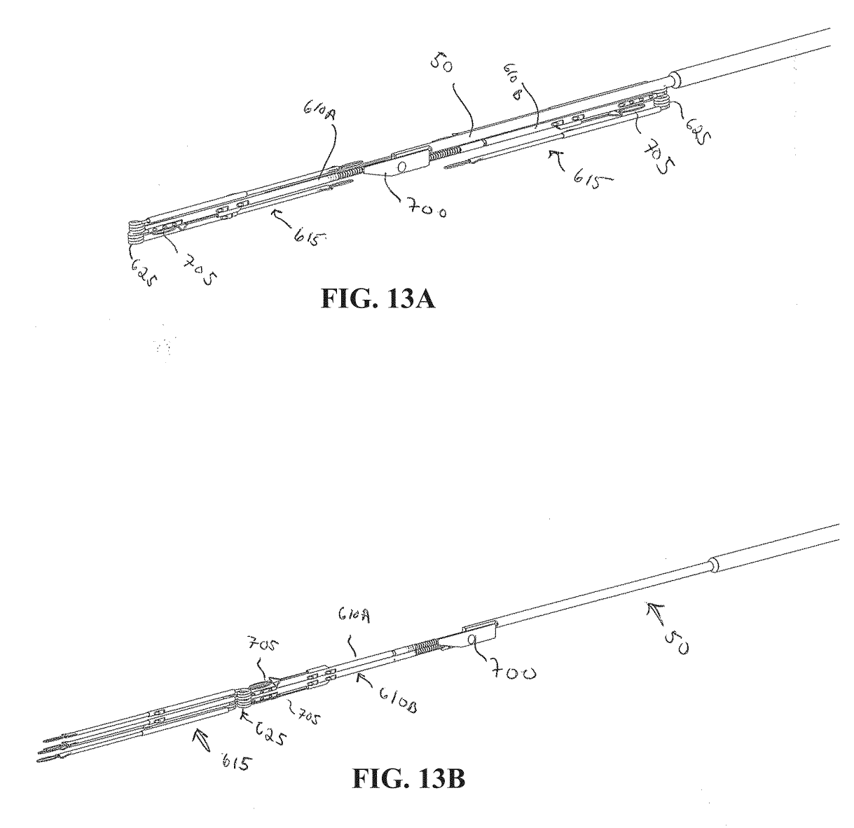

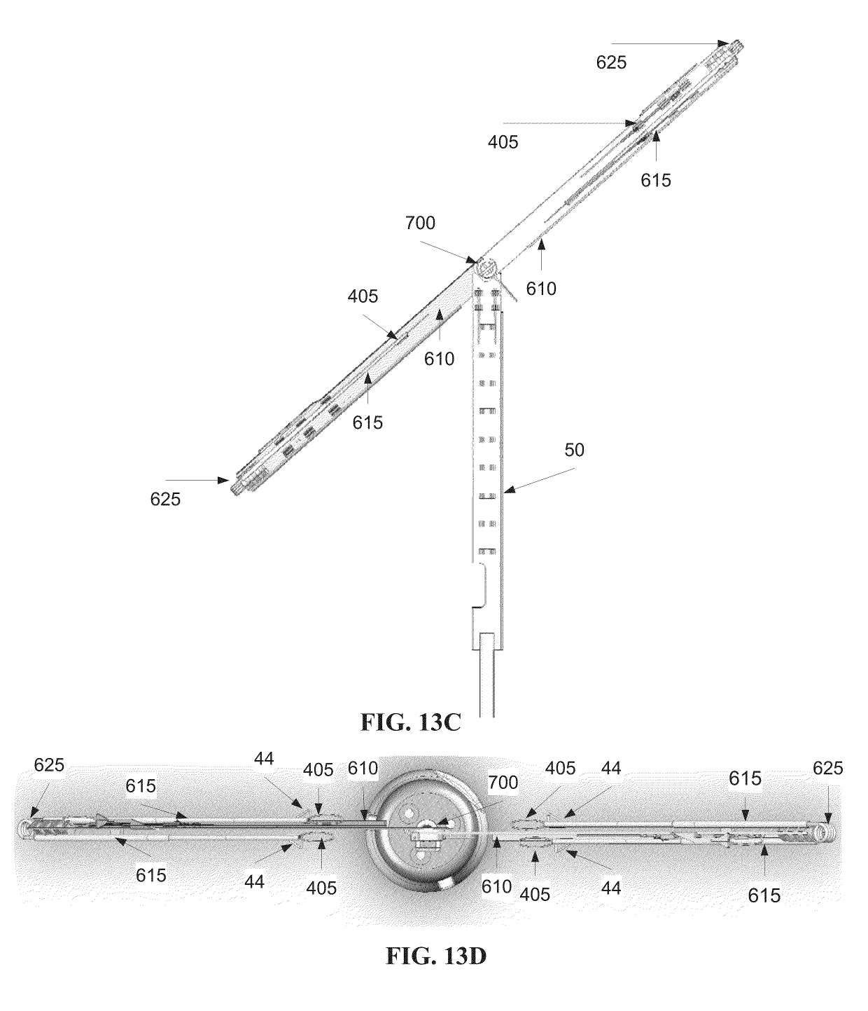

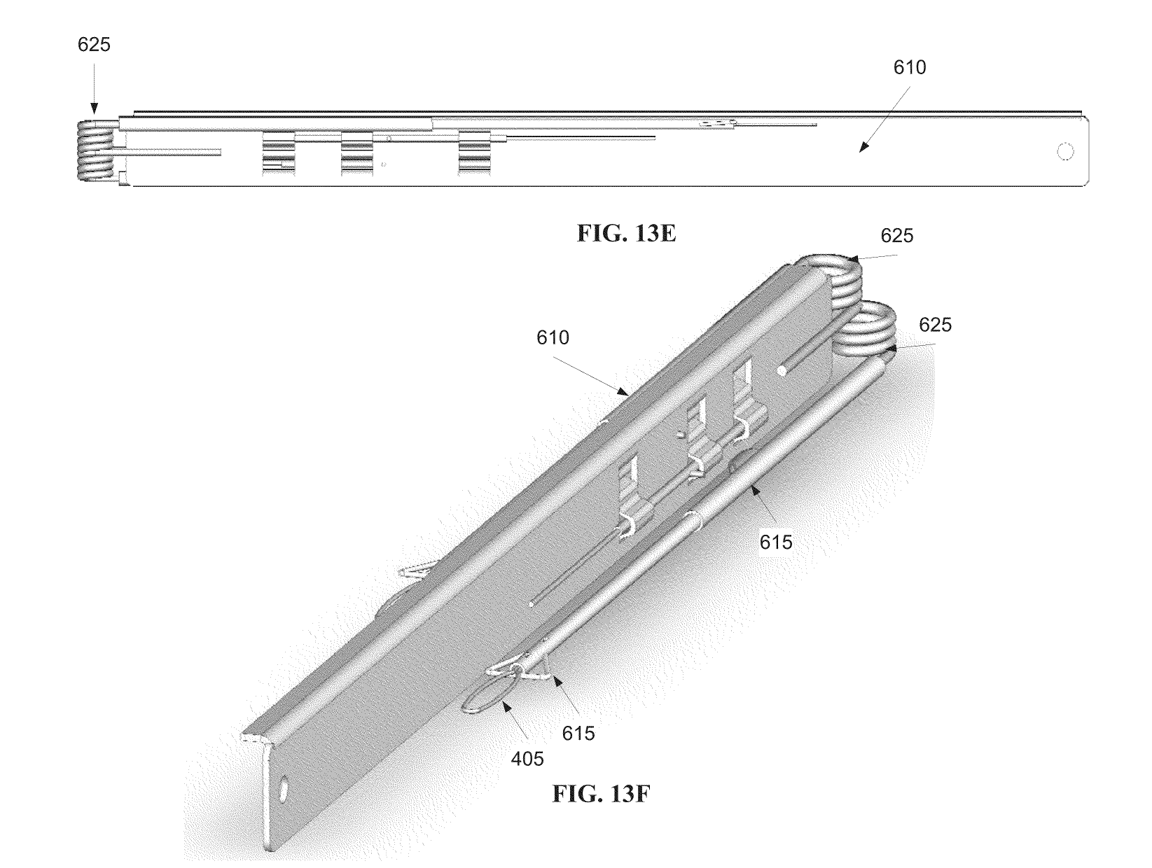

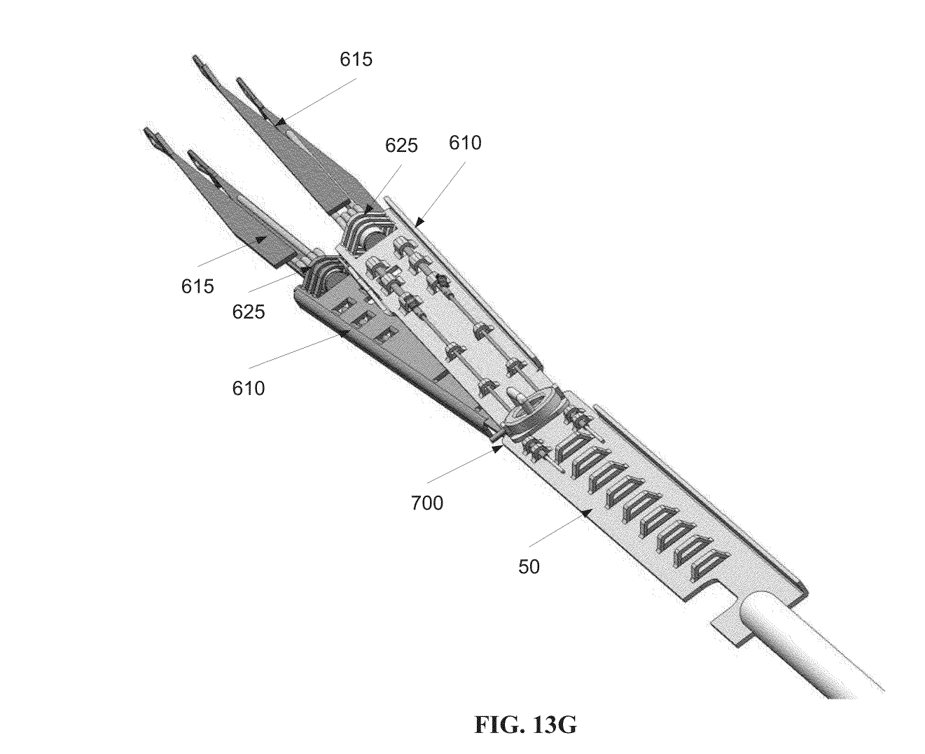

Reference is now made to FIGS. 13A and 13B showing simplified schematic illustrations of an exemplary guide rod with self-extending elements as positioned respectively in a delivery configuration and fully extended configuration for extraction through a delivery channel in accordance with some embodiments of the present invention. According to some embodiments of the present invention, during mesh delivery and mesh deployment, holding element 700 maintains supporting elements 610 from either side of guide rod 50 extended at a 180 degree angle. According to some embodiments of the present invention, after deployment and prior to extracting guide rod out of the body cavity, a locking mechanism in holding element 700 is released and supporting element 610A is folded away from guide rod 50 and toward supporting element 610B (FIG. 13B). Optionally, control pins 705 are connected to holding element 700, e.g. connected via a wire or string and provide for releasing the locking mechanism in holding element 705 in response to pulling control pins 705. Optionally, release of the locking mechanism in holding element 700 is controlled from a latch or button on a hand guiding unit 300.

Reference is now made to FIGS. 13C and 13D showing lateral and top simplified schematic illustrations of the guide rod 50 (exemplary) with the supporting elements 610 in a spread position and the self extending elements 615 in a folded position in accordance with some embodiments of the present invention. Reference is also made to FIGS. 13E and 13F showing simplified schematic illustrations of an exemplary supporting element 610 with self extending elements 615 in a folded position in accordance with some embodiments of the present invention. Reference is also made to FIGS. 13G showing a simplified schematic illustration of the guide rod 50 (exemplary) with the supporting elements 610 and the self extending elements 615 aligned substantially in parallel to the guide rod 50 in accordance with some embodiments of the present invention. The design depicted in FIGS. 13C-13G allows manufacturing the surgical mesh delivery and positioning system in a production line as the elements 50, 610, and 615 are provided with integrally formed niches, recesses, and spring holders. The elements 50, 610, and 615, which are made on plates, such as metal plates, polymeric plates, and/or alloy plates, can be formed using a mold. Such manufacturing may reduce the manufacturing cost of the surgical mesh delivery and positioning system.

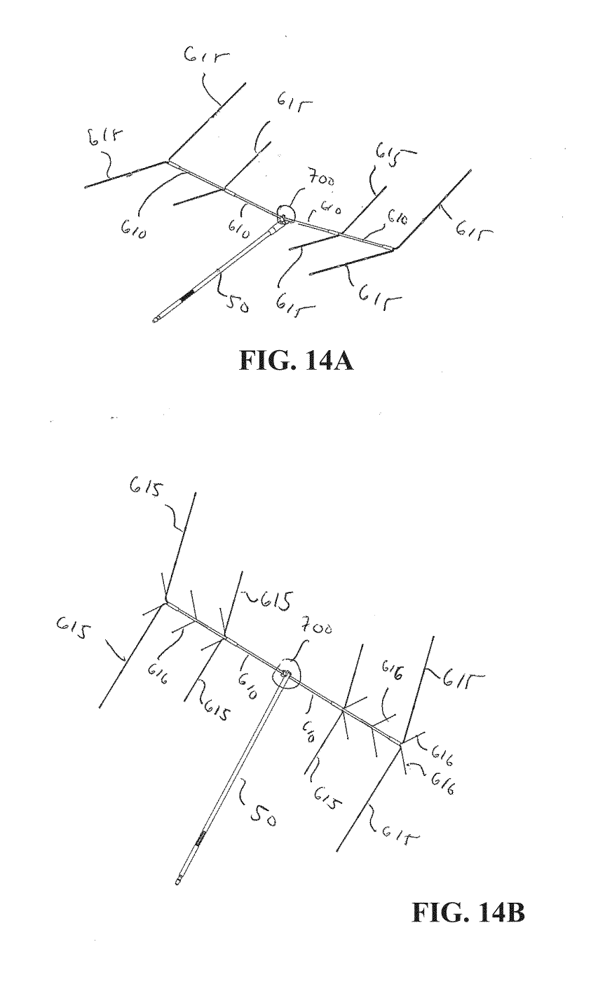

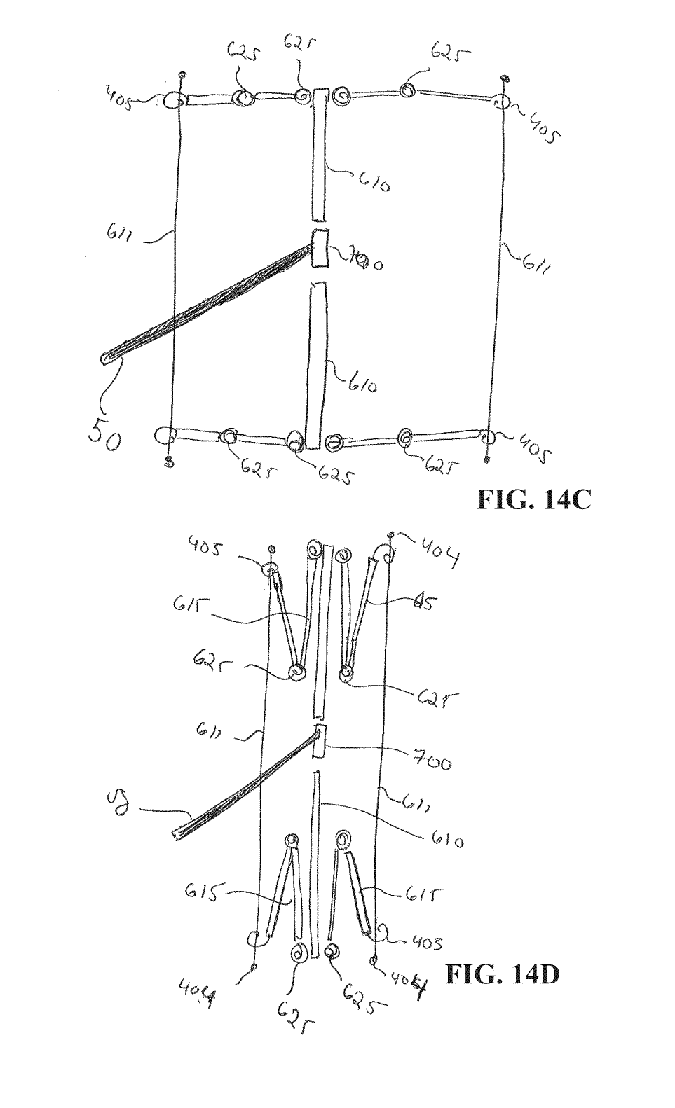

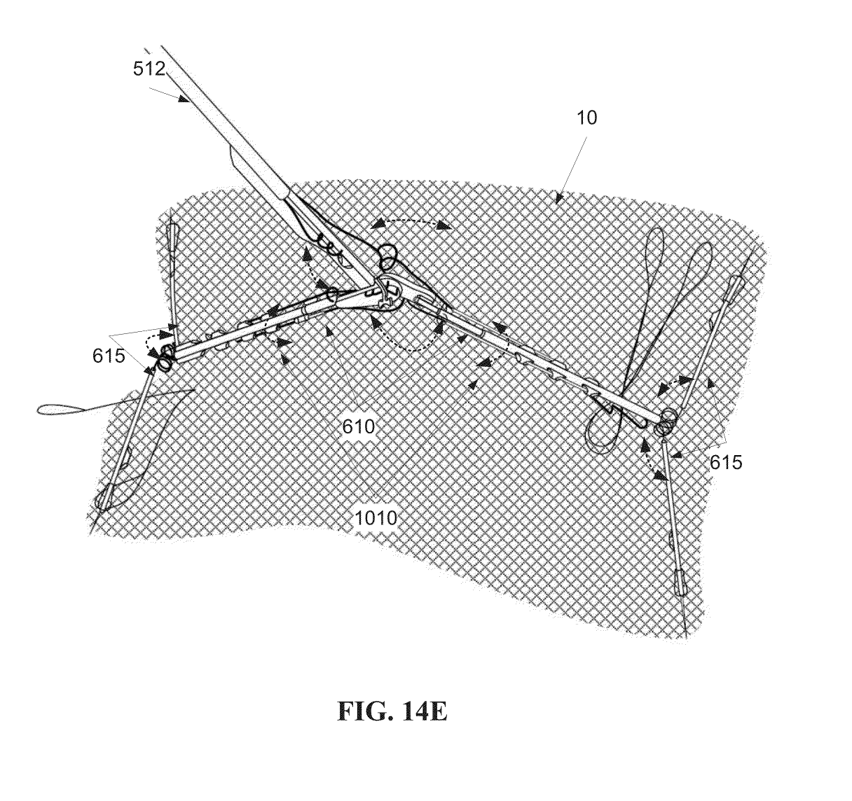

Reference is now made to FIGS. 14A and 14B showing alternate configurations of extending elements adaptable for larger scale meshes in accordance with some embodiments of the present invention. According to some embodiments of the present invention, guide rod 50 is connected to supporting elements 610 via holding element 700. In some exemplary embodiments, release of locking mechanism in holding element 700 provides for collapsing supporting elements 610 toward each other and away from guide rod 50 for extraction of the guide rode through a operative channel, e.g. channel 200(FIG. 1B). In some exemplary embodiments, prepackaged mesh unit 100 includes a plurality of elements 615 that extends from supporting element 610 during mesh deployment. Optionally elements 615 are elastic elements. Alternatively, elements 615 are supporting elements that are connected to elements 610 by an elastic connection. Optionally, prepackaged mesh unit 100 additional includes elements 616 extending from supporting element 610. Typically, elements 615 are shorter than elements 615 (and do not extend over the entire width of the mesh). Optionally, elements 616 are elastic provide for anchoring a mesh during fixation. Optionally, one or more elastic elements 616 are projected off of elements 615. Optionally, elastic elements 616 are projected from different angles with respect to supporting element 610.

Reference is now made to FIGS. 14C and 14D showing simplified schematic illustrations of an exemplary guide rod with self-extending unit including a plurality of elastic joints shown in a spread and folded configuration respectively in accordance with some embodiments of the present invention. According to some embodiments of the present invention, self-extending unit 733 includes supporting elements 610 that extend along the central line of the double cylinder scroll and a plurality of self-extending elements 615 connected by elastic joints 625. Typically, self-extending unit 733 is positioned in between the cylinders of a double scroll mesh in a prepackaged mesh unit 100 and operates to spread the mesh in response to releasing the collapsed self-extending elements 615. According to some embodiments of the present invention, ends of a series of extending elements 615 include a loop element 405 or a hook element through which an rod or wire 611 is fed through. Optionally, wire 611 includes stoppers 404 at either end to prevent wire 611 from slipping out of loops 405. Optionally, stoppers 404 have a smooth and/or flat surface adapted to slide over mesh as it is spread. According to some embodiments of the present invention, wire 611 flattens an associated mesh as self extending unit expands from a folded (or collapsed) configuration (FIG. 14D) to an expanded configuration (FIG. 14C).

Reference is now made to FIG. 14E showing alternate configurations of supporting elements 610 adaptable for matching to intrabody curved surfaced in accordance with some embodiments of the present invention. In these embodiments, the angle between the supporting elements 610 is less than 180°, for example between about 130° and about 140°. In these embodiments, the distance between the spread mesh and the tip of the guiding rod is at least 2 centimeters, for example between about 5 cm and about 25 cm. In such a manner, a gap is formed between the spread mesh 10 and the guide rod 50. Optionally, the elements 615 and/or supporting elements 610 are connected to elastic joints that increase the elasticity in a spread state. Such gap, and optionally elasticity, allows adapting the curvature of the mesh to the topography of a curved surface, for example the groin. The adaptation facilitates spreading a single mesh in a manner that covers non planner raptures, such as groin raptures. It should be noted that the dotted arrows indicate movement of elements 610, 615, and 50. It should be noted that the supporting elements 610 in this embodiment, or in any other embodiment may roll around its axis, as shown at 1010.

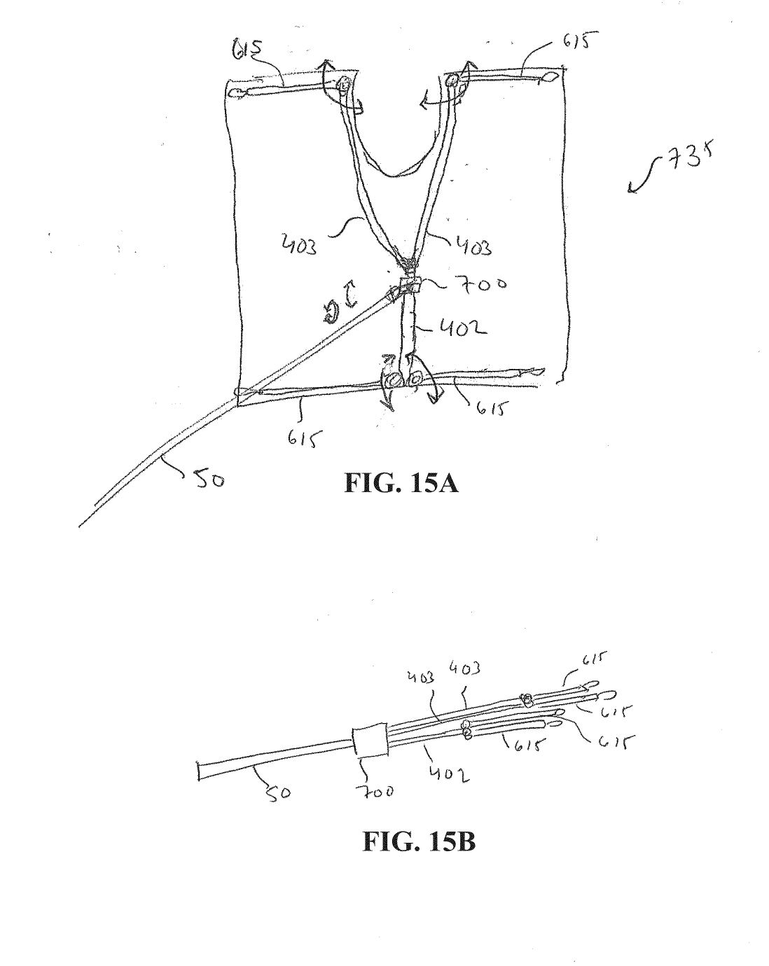

Reference is now made to FIGS. 15A and 15B showing a simplified schematic illustration of an exemplary self-extending unit adapted to be inserted into a partially rolled horseshoe mesh using a horseshoe double cylinder scroll method, shown respectively in a full extended and collapsed configuration in accordance with some embodiments of the present invention. According to some embodiments of the present invention, self extending unit 735 provides for deploying a horse shoe mesh without requiring rolling of the mesh together with the self-extending elements, e.g. similar to the configuration described in reference to FIGS. 12-13. According to some embodiments of the present invention, during delivery, elements 615 are folded in toward elements 402 positioned in a center line of mesh 10 and 403 in a V-shape around opening 428 and during deployment of mesh 10, elements 615. In some exemplary embodiments, guide rod 50 is connected to elements 403 and 402 via holding element 700. Typically, connection of guide rod is rotatably connected to holding element 700. Typically, guide rod can rotate with respect to holding element 700 so that the rod can alternately be positioned perpendicular to the surface of the mesh and parallel to the surface of the mesh when and to element 402. Optionally, the connection between guide rod 50 and holding element 700 provides for rotation of guide rod 50 about central longitudinal axis of element 402. In some exemplary embodiments, holding element 700 includes a locking mechanism that locks elements 402 in a 180 angle with respect to elements 403 while in a locked position. Referring now to FIG. 15B, in some exemplary embodiments, releasing of the locking mechanism of holding element 700 (typically after deployment of the mesh) allows elements 402 to fold toward elements 403, so that the self-extending unit can be compactly pulled out of the body cavity through an operating channel through which guide rod 50 is manipulated. Optionally, the holding device includes a joint that is connected, for example via shafts which are placed in parallel to the guide rod 50, to a maneuvering handle. This allows the user to adjust the angle of the holding element 700 so as to tilt elements 403 and 402. This tilting maneuver the mesh 10 which is connected to elements 403 and 402. Such tilting facilitate the placing procedure as it allows the user to control the angle of the mesh before the release thereof, according to the orientation and/or location of the target rapture.

It is noted that although flexible joint 59 (FIG. 6B) has been described in reference to embodiments describing a prepackaged mesh unit adapted for an umbrella fold, guide rods and prepackaged mesh units adapted for other types of folds and hernias may also include a flexible joint 59 for improved access and flexibility.

It is similarly noted that although self-extending elements including a rigid covering, with flexible connections constructed from a coil or a bundle of elastic wire and with optional channels for accommodating locking pins have been mostly described in reference to specific embodiments or folds similar extending elements may be used with other types of folds described herein.

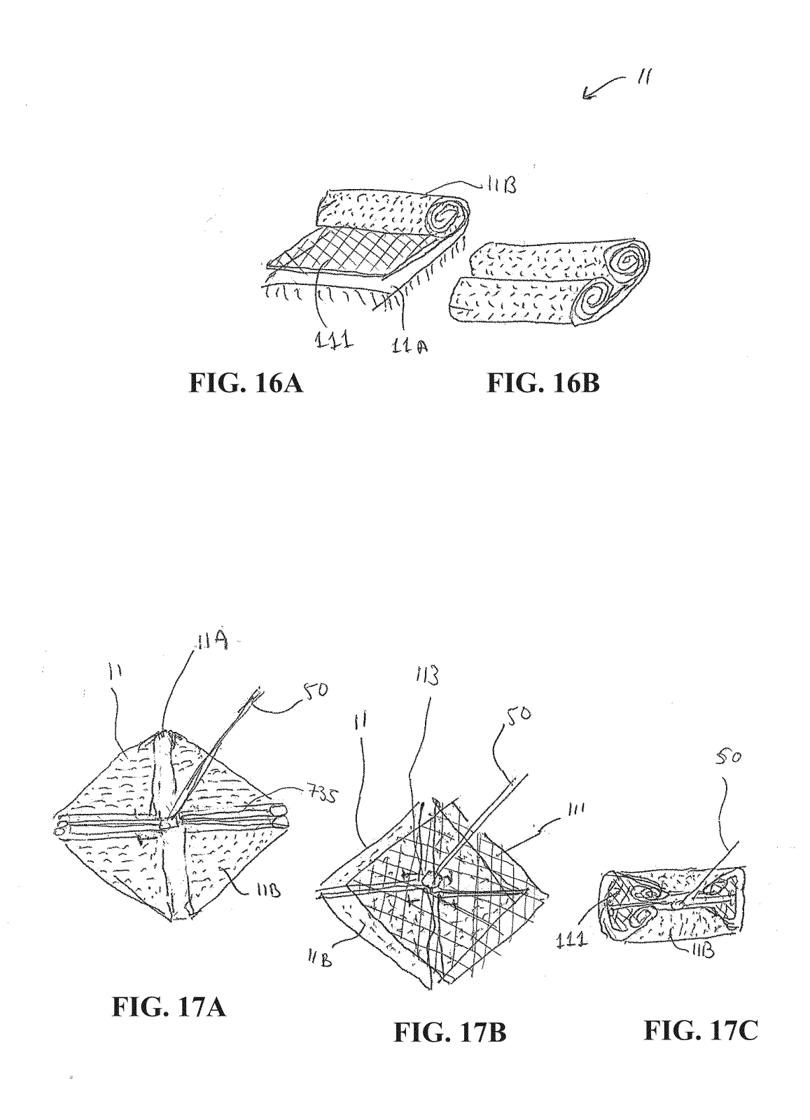

Reference is now made to FIGS. 16A and 16B showing simplified schematic illustrations describing a packaging configuration that may be used for collapsing a self-adhesive mesh in accordance with some embodiments of the present invention. According to some embodiments, a self-adhesive mesh 11 is rolled and/or folded prior to delivery into the body cavity. Typically, mesh 11 includes an adhesive side 11B and a non-adhesive side 11A. In some exemplary embodiments of the present invention, a protective cover 111 is positioned over the non-adhesive side of the mesh prior folding to provide protection against self-adhesive mesh 11 sticking to itself during packaging and self-deployment. In some exemplary embodiments, protective cover is made from a thin sheet of polypropylene. Optionally, protective cover 111 is transparent and/or translucent so that the mesh can be seen through cover 111. According to some embodiments of the present invention, self-adhesive mesh 11 with protective cover 111 is delivered and deployed with a prepackaged mesh unit. Optionally, self-adhesive mesh 11 is packaged in a double cylinder scroll fold. Optionally, self-adhesive mesh 11 is packaged in a single cylinder scroll fold, e.g. single roll.

Reference is now made to FIGS. 17A, 17B and 17C showing simplified schematic illustrations describing an alternate packaging configuration that may be used for collapsing a self-adhesive mesh in accordance with some embodiments of the present invention. In some exemplary embodiments, prior to rolling a mesh, e.g. self-adhesive mesh 11, the corners of mesh 11 are folded in toward the center of the mesh to form a diamond fold (FIG. 17A). In some exemplary embodiments, the mesh is folded with a self-extending unit, e.g. self-extending unit 734 (FIG. 12A) attached to a guiding rod 50 and the corners of the mesh are attached to ends of self-extending elements, e.g. elements 615. In some exemplary embodiments, when using a self-adhesive mesh 11, a protective cover 111 is positioned over the diamond fold so that the corners of the diamond fold can be rolled toward the center of the mesh in a double cylinder scroll fold without the mesh sticking to itself. In some exemplary embodiments, protective cover 111 includes an opening 113 so that it can be fitted through guide rod 50. In some exemplary embodiments, protective cover 111 is attached to guide rod 50 and is removed from the body cavity together with guide rod 50 at the termination of mesh deployment.

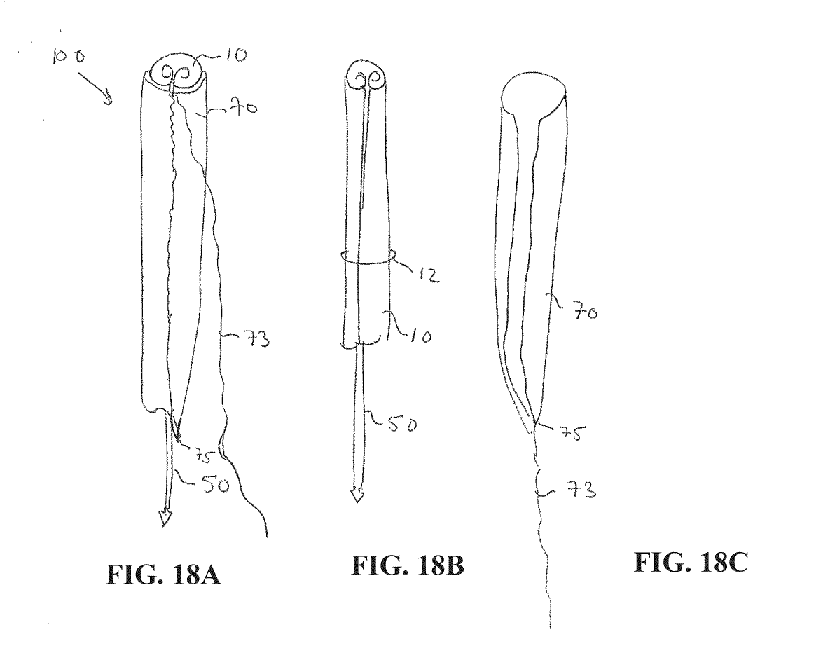

Reference is now made to FIGS. 18A, 18B and 18C showing simplified schematic illustrations of an exemplary removable sheath and underlying packaged mesh in accordance with some embodiments of the present invention. According to some embodiments of the present invention, prepackaged mesh unit includes a protective sheath 70 wrapped around mesh 10. Optionally, sheath 70 provides a hermetic seal. Optionally, sheath 70 is transparent so that the mesh can be viewed through the sheath. Optionally, sheath 70 is constructed from a smooth material so that prepackaged mesh unit can be passes through operative channel 200 with relative ease. According to some embodiments of the present invention, sheath 70 is removed during the delivery process, e.g. as it is passed through operative channel 200. Optionally, sheath 70 is removed either before or after delivery. According to some embodiments of the present invention, sheath 70 includes a string 73 that can be used to tear the seal and expose the packaged mesh 10. In some exemplary embodiments, string 73 is fixated on the sheath at a point 75 on the sheath and is further used to retract the sheath out of the body cavity.

In some exemplary embodiments, sheath 70 is constructed from an elastic material and compresses packaged mesh 10 when positioned over and/or around it. Optionally, sheath 70 is applied on mesh 10 prior to delivery so that mesh 10 is not damaged by long term compressive forces. The present inventor has found that the amount of compression applied on the mesh without causing damage to the mesh can be significantly increased when the compressive forces are applied over a relative short term period. Comparable compressive forces applied during packaging at a manufacturing site and maintained over a storage period, e.g. lasting a few months to about two years may cause irreparable damage to the mesh. Compressing prepackaged mesh unit to a minimal diameter is especially important for NOTES application where the channel through which the mesh can be introduces has limited diameter.

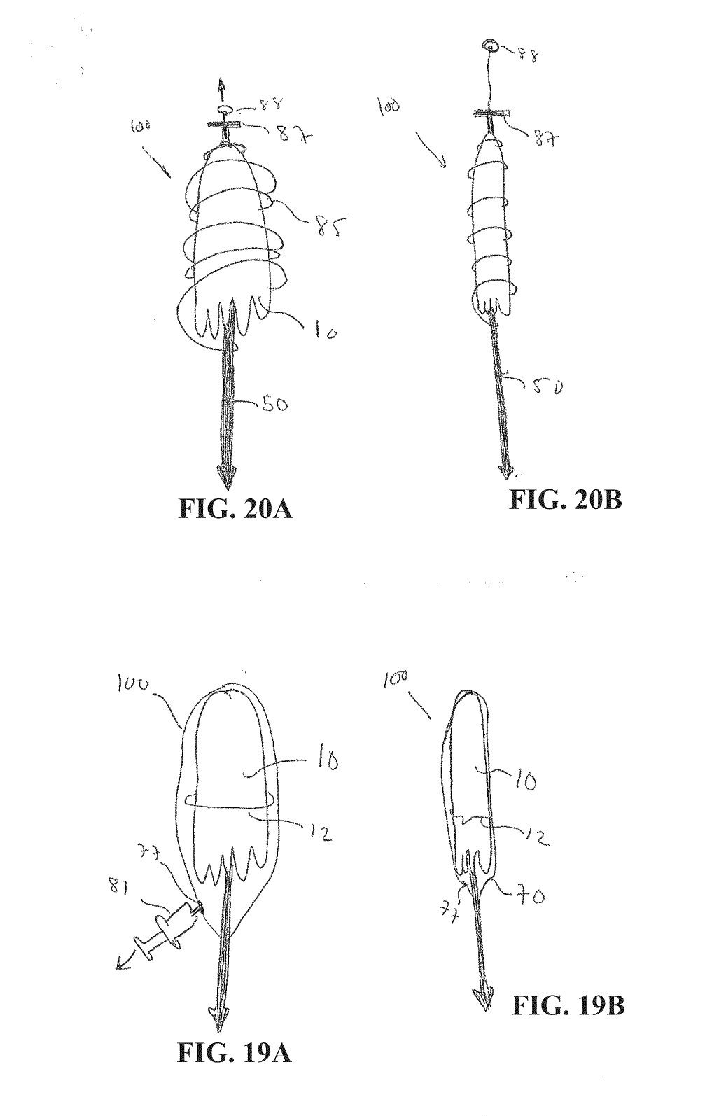

Reference is now made to FIGS. 19A and 19B showing a simplified schematic illustrations describing an exemplary method for vacuum packing the prepackaged mesh prior to delivery in accordance with some embodiments of the present invention. According to some embodiments of the present invention, sheath 70 provides a hermetic seal. In some exemplary embodiments, sheath 70 includes a valve 77, e.g. a one way valve for removing air from prepackaged mesh unit 100. In some exemplary embodiments, air is removed just prior to delivery to avoid damage to the mesh as a result of prolonged compressive forces exerted on the mesh. In some exemplary embodiments a syringe 81 is used to vacuum pack the mesh to a desired volume. It is noted that the different compressive methods can be applied any of the packaging methods, e.g. umbrella fold and double cylinder scroll fold.

FIGS. 20A and 20B are simplified schematic illustrations describing an exemplary method for compressing the prepackaged mesh prior to delivery in accordance with some embodiments of the present invention. According to some embodiments of the present invention a string or band is spirally wrapped around mesh 10 or a sheath 70 (not shown) covering mesh 10 and is tightened to compress the volume occupied by prepackaged mesh unit 100. Optionally string 85 is fixated on guide rod 50 on one end and fed through a channel 87 positioned at the head of packaged mesh 10 on an opposite end. Optionally string 85 has a looped end 88 from which the string can be pulled. In some exemplary embodiments, loop 88 is fed through operative channel 200 and pulled to compress prepackaged unit 100 while pulling it into operative channel 200. Typically, the inner diameter of operative channel 200 closely matches a diameter of prepackaged mesh unit in a compressed state. In some exemplary embodiments, string 85 is removed after insertion into operative channel 200. Optionally, after insertion through operative channel 200, loop 88 and channel 87 is removed and string 85 is pulled out of the channel from its opposite end fixated on guide rod 50.

In some exemplary embodiments, a combination of methods, e.g. more than one method is used to compress prepackaged mesh unit to a minimum diameter.

The Operative Channel

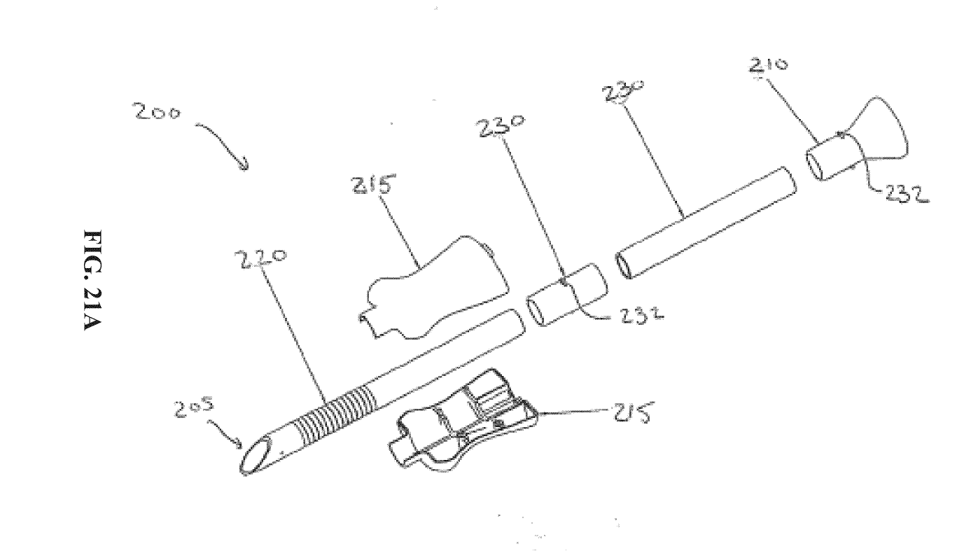

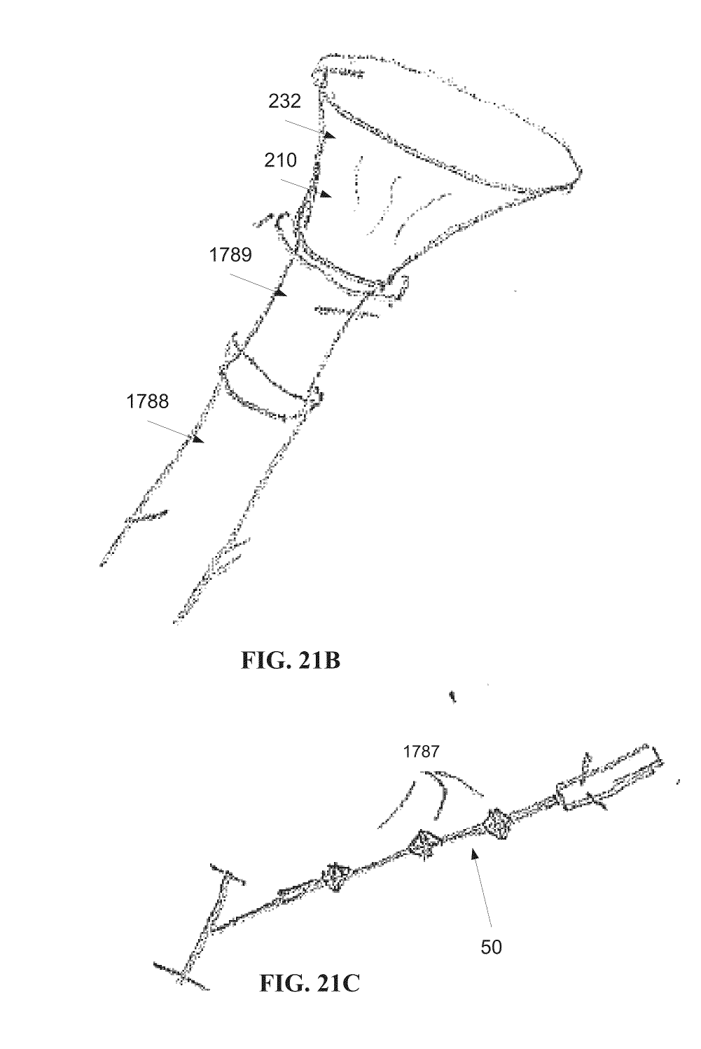

Reference is now made to FIG. 21A showing an exemplary operative channel in accordance with some embodiments of the present invention. According to some embodiments of the present invention, operative channel 200 is a generally cylindrical element that is constructed from a plurality of connecting parts that can be easily assembled and disassembled to adapt the channel for different functions and operations. In some exemplary embodiments a first end 205 of operative channel 200 is adapted for insertion through a port site and an opposite end 210 is adapted for easy introduction of a prepackaged mesh through the operative channel. Optionally, operative channel 200 is a single use or disposable device. Optionally, operative channel is transparent so that it does not obstruct viewing of the mesh. Typically, channel 200 has a smooth inner surface that aids in inserting prepackaged mesh unit 100 into operative channel 200 and delivering the mesh through the channel. According to some embodiments of the present invention, prepackaged mesh unit 100 is inserted into channel 200 before the channel is positioned in-vivo. Optionally the channel has a diameter that is smaller than the diameter of prepackaged mesh unit 100 and is used to further compress prepackaged mesh unit 100 as it is introduced through the channel. In some exemplary embodiments, the channel is introduced through a trocar and slanted opening 205 is not required.

Optionally a valve and/or plug is inserted at and end distal to end 205 and used to maintain abdominal cavity pressure during a procedure.

Typically, the diameter of operative channel 200 is adapted, e.g. chosen according to the size of prepackaged mesh unit. This is of particular importance to laparoscopic and/or endoscopic procedures that aim to reduce the exposure area of the procedure. According to some embodiments of the present invention, the length of the channel is adapted to the length of the prepackaged mesh. Optionally, one or more extension units 230 that fit into each other can be used achieve the desired channel length. Optionally the extension elements include prongs 232 that limit the motion of the extension through the channel. In some exemplary embodiments, extensions 230 are used to deliver a mesh and then subsequently removed once the mesh is delivered so that channel 200 can function as an operative trocar through which surgical instruments can be introduced and manipulated. According to some embodiments of the present invention, grip handle 215 is used as a handle to manipulate operative channel 200 and also as a safety feature to prevent excessive slippage of channel 200 into the body cavity. Optionally, ribs 220 also function to reduce the amount of slippage.

According to some embodiments of the present invention, funnel opening 210 helps to introduce prepackaged mesh unit through operative channel 200.

According to some embodiments of the present invention, for example as depicted in FIG. 21B, an adaptor which includes the funnel opening 210, the prongs 232, and a trocar connection element 1789 that is designed to be inserted into an existing trocar 1788 is provided. The trocar connection element 1789 is optionally a tube having a diameter that is adjusted to the inner diameter of the channel of the trocar 1788. The adaptor allows using existing trocar for performing the placing of the mesh using the aforementioned surgical mesh delivery and positioning system, for example the delivery and positioning system depicted in FIG. 12A. The adaptor with the funnel opening 210 and the prongs 232 facilitates the insertion of the surgical mesh delivery and positioning system into the trocar. It should be noted that placing the adaptor and the surgical mesh delivery and positioning system in an existing trocar may cancel the operation of valves for maintain abdominal pressure. Optionally, a plurality of valves for maintaining abdominal pressure, such as silicone ring valves, are positioned on the release guide rod 50 to replace the cancelled valves, for example as depicted by numeral 1787 of FIG. 21C.

The Hand Guide Unit

Reference is now made to FIG. 22 showing an exemplary hand guiding unit in accordance with some embodiments of the present invention. According to some embodiments of the present invention, hand guide unit 300 is operable to latch on to and release guide rod 50 from end 301 while holding handle grip 320 and controlling locking and triggering with knobs 332 and 331 respectively from a distal end. In some exemplary embodiments, spring element 351 is adapt to catch and hold guide rod 50 along its receding diameter 54 (FIG. 2A) and prongs 352 are adapted to catch spokes 57 and prevent rotation of guide rod 50 with respect to rod 310 (FIG. 2A). According to some embodiments of the present invention locking knob 332 controls lifting and lowering spring element 351 to release and latch guide rod 50. Optionally, markings on knob 332 indicate the locking state of the hand guide unit, e.g. indicating a lock or release state. According to some embodiments of the present invention triggering knob 331 controls inner rod 311 that can push out guide rod 50 once released. In some exemplary embodiments, rod 310 includes a marking that clearly indicates when the guide rod is released.

In some exemplary embodiments, hand guide 300 replaces the guide rod so that the self-extending elements are fixated directly on an end of hand guide 300 distal from hand 320. Optionally, hand guide 300 includes one or more hand control, e.g. buttons or knobs providing mechanical control for remotely releasing one or more of the self-extending elements.

Exemplary Methods for Mesh Delivery and Positioning for Specific Applications

Laparoscopic procedures

According to some embodiments of the present invention, the mesh is delivered and positioned during a laparoscopic surgical procedure.

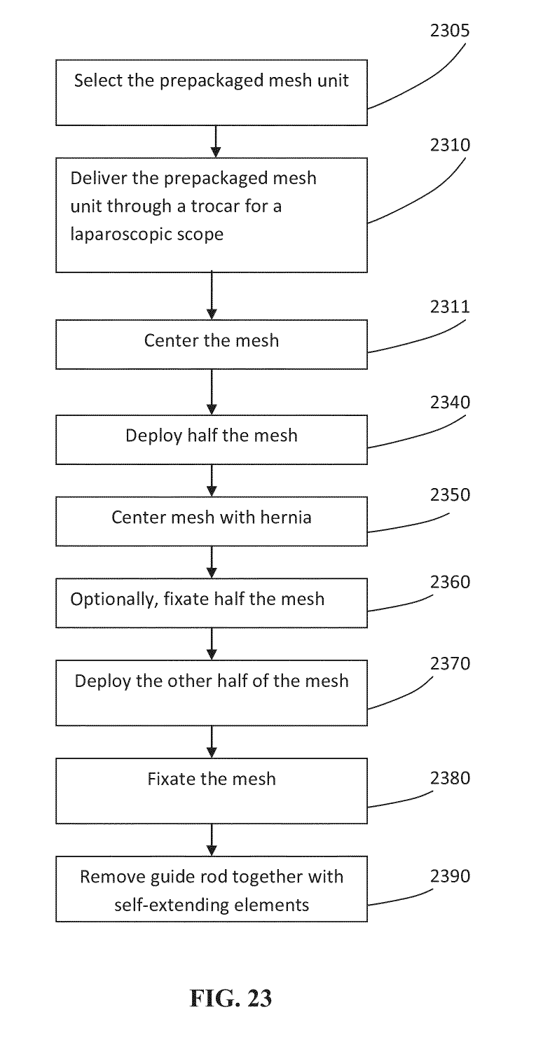

Reference is now made to FIG. 23 showing a simplified flow chart describing an exemplary method for delivering and positioning a mesh during a laparoscopic procedure in accordance with some embodiments of the present invention. According to some embodiments of the present invention, a prepackaged mesh unit is selected based on the size and type of mesh required (block 2305). In some exemplary embodiments, prepackaged mesh unit 100 is delivered through the trocar housing the laparoscopic scope which is generally the largest diameter trocar. Optionally, an alternate port site or trocar can be used to deliver prepackaged mesh unit 100. In some exemplary embodiments, the operative channel together with the prepackaged mesh unit is inserted through the trocar to deliver the mesh. Optionally, the trocar is removed and operative channel 200 is position in place of the trocar. In some exemplary embodiments, the trocar is removed to deliver a mesh have a diameter larger than the trocar inner diameter. Optionally the port site incision can be increased to accommodate a larger size prepackaged mesh unit.

According to some embodiments of the present invention, the hand guide unit is used to push the prepackaged mesh unit through the operative channel and/or trocar and deliver the prepackaged mesh unit in-vivo (block 2310). Subsequently, in some exemplary embodiments (block 2311), the tip of the guide rod 50, which is connected to the one or more supporting elements 610, is placed approximately in front of the center of a deployment area, such as a rupture (i.e. hernia) which is about to be covered by the mesh. This placing procedure assures that the mesh is spread to cover completely the deployment area. In some exemplary embodiments, the hand guide unit is disconnected from the prepackaged mesh unit 100, e.g. disconnected from guide rod 50 and is removed from the trocar so that the laparoscopic scope can be replaced and used for position, releasing and fixating the mesh. Optionally, the mesh is not fixated during this procedure.

According to some embodiments of the present invention, the hand guide can be reinserted through an alternate, generally narrower trocar and/or port site and re-engaged with guide rod 50 to position and anchor the prepackaged mesh unit with the aid of the scope. Optionally a surgical tool can be used to help re-engage the hand guide unit with guide rod 50. In some exemplary embodiments, once the prepackage mesh unit is positioned in the herniated area; one half of the mesh is deployed (block 2340). Typically, once half of the mesh is deployed, the surgeon aligns the center of the mesh with the center of the hernia (block 2350). Opening only half the mesh provides for increased visibility during centering of the mesh, e.g. the part of the mesh that is still collapsed does not obstruct view of the herniated area. Optionally, the deployed half of the mesh if fixated, e.g. with staples (block 2360). Optionally, once the mesh is centered, the other half of the mesh is deployed (block 2370) and the mesh is fixated (block 2380). Guide rod 50 together with the self-extending elements and optionally other elements included in the prepackaged mesh unit other than the mesh are removed from the body cavity with hand guide unit 300 (block 2390). Optionally some tension is applied during fixation, for example, by suturing tensioned muscle to mesh

Groin (Inguinal) Hernia

Typically for groin hernia repair procedure rectangular meshes of size 10—13 cm to 15—15 cm are used. According to some embodiments of the present invention, the mesh is rolled in a vertical double cylinder scroll fold together with 6-8 self-extending elements 400. Optionally, the self-extending elements are in the form of super-elastic bands that are stronger in the vertical axis (with respect to the vertical direction of the double cylinder scroll). Typically, the scroll is clasped with at least two clasps 12 or locking pins 409 so that each scroll can be separately unrolled, e.g. extended. In some exemplary embodiments, operative channel 200 having an inner diameter of around 6-8, e.g. 7 mm and an outer diameter of between 8-10, e.g. 9 mm is used to deliver the prepackaged mesh. In some exemplary embodiments, the length of the channel is around 10-12 cm.

Once the prepackaged mesh unit is delivered, hand guide unit 300 positions prepackaged mesh unit 100 is positioned and vertically centered over the herniated region. Clasp(s) 12 are or locking pins 409 released to unravel one of the scrolls while the prepackaged mesh unit is supported or slightly pushed against the herniated wall. Self extending elements 400 once extended can be further used to slide, e.g. pan the mesh to a desired location along the wall. In some exemplary embodiments, once positioned the unraveled part of the mesh is fixated by suturing or stapling while self-extending elements flatten the mesh against the herniated wall. In some exemplary embodiments, the second cylinder scroll is released and secured while self-extending elements flatten the mesh against the herniated wall. Once fixated, hand guide unit 300 can retract guide rod 50 through channel 200 out of the body cavity.

Ventral (Umbilical) Hernia

Typically for ventral (umbilical) hernia repair procedures, a round or elliptical mesh of up to about 22 cm in diameter is used. According to some embodiments of the present invention, the mesh is rolled in a vertical double cylinder scroll fold together with 6-8 self-extending elements 400. Optionally, the self-extending elements are in the form of super-elastic bands. The mesh can be rolled or collapsed using a horizontal double cylinder scroll or an umbrella fold. When using a horizontal double cylinder scroll the vertical axis bands are stronger.

Typically, positioning of the mesh is performed through trocars in the flank area. As described above with respect to groin hernia repair, the packaged mesh is centered with the hernia and released, e.g. one side at a time. In some exemplary embodiments, when an oval shaped mesh is used in an umbrella fold, the horizontal axis of the elliptical mesh is marked.

Hiatal Hernia

Typically for Hiatal hernia repair procedures a horseshoe shaped mesh of sizes between 10×10 cm and 10×14 cm is placed above the Hiatus area behind the stomach and around the esophagus. Typically, the opening of the mesh is placed on the bottom part of the esophagus, behind the stomach, and the cylinders are at both sides of the esophagus. According to some embodiments of the present invention, a mesh for a Hiatal hernia is rolled in a vertical double cylinder scroll with about 4 self-extending elements 400, e.g. 4 super-elastic bands. Typically, each cylinder is secured with a clasp 12. According to some embodiments of the present invention, the right cylinder is released and fixated first followed by the left one. Alternatively both cylinders are released prior to fixation.

### Post Operative Incisional Hernia (PIOH)

Typically in PIOH repairs the mesh is elliptic and can reach a vertical axis of about 34 cm. In some exemplary embodiments, 8-10 self-releasing elements are used optionally constructed from super-elastic bands. The mesh can be packaged in an umbrella fold or a double cylinder scroll. In some exemplary embodiments, positioning of is similar to the method described in reference to the vertical hernia repair.

Trans Abdominal—Umbilical or Inguinal or Trocar site Hernia Procedures

Typically, trans-abdominal procedures are performed for small hernias of up to 2 cm in diameter. The mesh is either round, e.g. for umbilical hernia with a diameter of about 10 cm; or elliptic, e.g. for groin hernia with a major axis of about 12 cm. According to some embodiments of the present invention, for trans-abdominal procedures, the mesh can be folded in an umbrella fold or a double cylinder scroll roll with about 6 self-extending elements, e.g. super-elastic bands. Typically, in small groin hernias a horizontal double cylinder scroll is used with an elliptical mesh. Circular meshes are typically used to repair small umbilical hernias and trocar site hernias. According to some embodiments of the present invention, an umbrella fold is used for packaging the circular mesh.

Typically, in trans-abdominal procedures the mesh is required to open in a cavity as opposed to against a wall. According to some embodiments of the present invention, the self-extending elements of the prepackaged mesh unit are secured to the mesh with short pieces of mesh material positioned on the surface of the mesh facing the abdominal wall (FIGS. 5A and 5B) and self-extending elements provide rigidity to the mesh. In some exemplary embodiments bands 510 or pockets 520 are placed about 1.5 cm from the edge of the mesh and are used to hold the mesh while opening the mesh within the cavity. Once the mesh is opened and secured, the self-extending elements can easily slide off the mesh by simply retracting the guide rod with self-extending elements out of the body cavity.

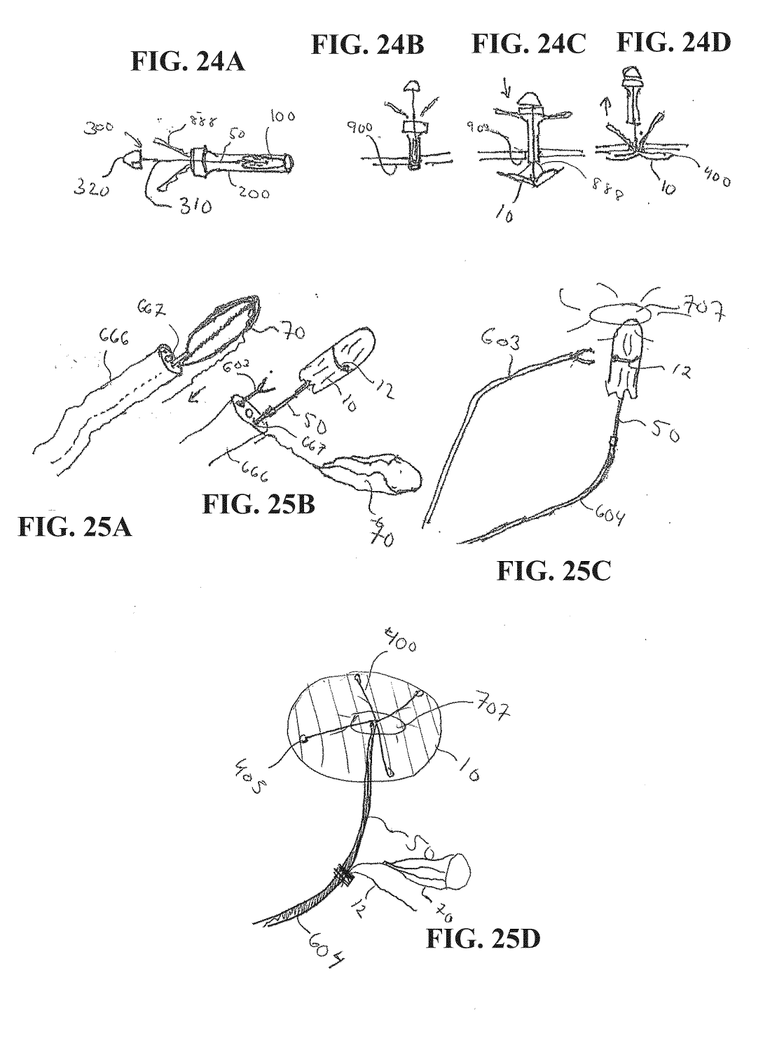

Reference is now made to FIGS. 24A, 24B, 24C and 24D showing simplified schematic illustrations describing an exemplary method for delivering and positioning a mesh during a trans-abdominal umbilical hernia operation or a intrabody rapture surgery wherein the mesh is guided toward the body via a natural orifice in the body, such as a vaginal hernia (via the vaginal lumen), stomach hernia (via the esophagus), and colon hernia (via the rectum) in accordance with some embodiments of the present invention. According to some embodiments of the present invention, a hand guide unit 300 with a small handle grip 320 and short handle rod 310 is used for trans-abdominal procedures. According to other embodiments of the present invention, an endoscope is used for intrabody rapture surgeries. According to some embodiments of the present invention, prepackaged mesh unit is inserted into channel 200 or a tip of an endoscope and latched onto hand guide unit 300 or onto a respective unit at the tip of the endoscope. Typically, channel 200 or the tip of the endoscope which is set to hold the prepackaged mesh unit 100 has a length that corresponds to the length of prepackaged mesh unit 100.

Optionally, plurality of string elements 888 are connected to the mesh on one end and extend out of channel 200 or the tip of the endoscope on the other end. In some exemplary embodiments, string elements 888 are used to hold the mesh from the outside while the mesh is opening and to pull the mesh toward the herniated area. Optionally, an additional string (not shown) is connected to a band 12 placed around the mesh and extends out of channel 200 or the tip of the endoscope and is used to release the mesh when pulled. Optionally, a first half, e.g. a semicircle of mesh 10 is released while the surgeon positions the mesh at a center of the herniated area as can be clearly viewed from the half of the hernia that is not covered by the mesh. Once the mesh is centered, the other half of the mesh can be released and positioned over the rest of the hernia. Alternatively, mesh 10 expands once it exits through channel 200 into the abdominal cavity or the tip of the endoscope to any other intrabody cavity. According to some embodiments of the present invention, two plastic tubes are added to the prepackaged mesh unit. Typically, the tubes are positioned alongside the self-extending elements and extend out of channel 200 or the tip of the endoscope. In some exemplary embodiments, the tubes can be used to inject biological glue to fixate the mesh.

According to some embodiments of the present invention, channel 200 is inserted directly to the abdominal cavity, or the endoscope to any other intrabody cavity, though the hernia defect 900. Optionally, a string controlling a band 12 around mesh 10 is pulled to release mesh 10. In some exemplary embodiments, strings 888 are pulled as mesh 10 extends to provide for fixating the mesh on the herniated wall. Optionally, biological glue is injected through the tubes and used to fixate the mesh on to the herniated wall. Once fixated, the hand guide unit together with the guide rod and self-extending elements are pulling out through channel 200 or the tip of the endoscope.

In some exemplary embodiments, a similar method for delivering and positioning a mesh is used for spreading a double cylinder scroll mesh during a groin (inguinal) hernia procedure.

### NOTES Procedures

Reference is now made to FIGS. 25A, 25B, 25C and 25D showing simplified schematic illustrations describing an exemplary method for delivering and positioning a mesh as part of a natural orifice transluminal endoscopy procedure in accordance with some embodiments of the present invention. According to some embodiments of the present invention, a prepackaged mesh unit is attached to the tip of a flexible rod inside a channel 667 of an endoscope 666, and thus is led into the abdominal cavity. According to some embodiments, sheath 70 is a hermetic sheath that protects the whole mesh from gastric secretion on the way to the abdominal cavity. According to some embodiments of the present invention, the prepackaged mesh unit is directed with the flexible rod 604 to the hernia area 707 where the sheath is removed. In some exemplary embodiments, the sheath is removed by pulling of a string but the sheath and string remain fixated to the endoscope so that it can be removed. Optionally, a surgical tool 603 is used to release the sheath and/or release a band 12 around the mesh 10. Fixation of the mesh can be performed with biological glue injected through accompanying tubes or by stapling.

It is noted that although the present system and methods have been described mostly in reference to mesh in-vivo delivery and positioning, the same or similar methods can be applied to in-vivo delivery and positioning of other materials such as biological sheet material and patches and are within the scope of the present invention. It is also noted that the system and methods described herein can be used for human as well as animal procedures.

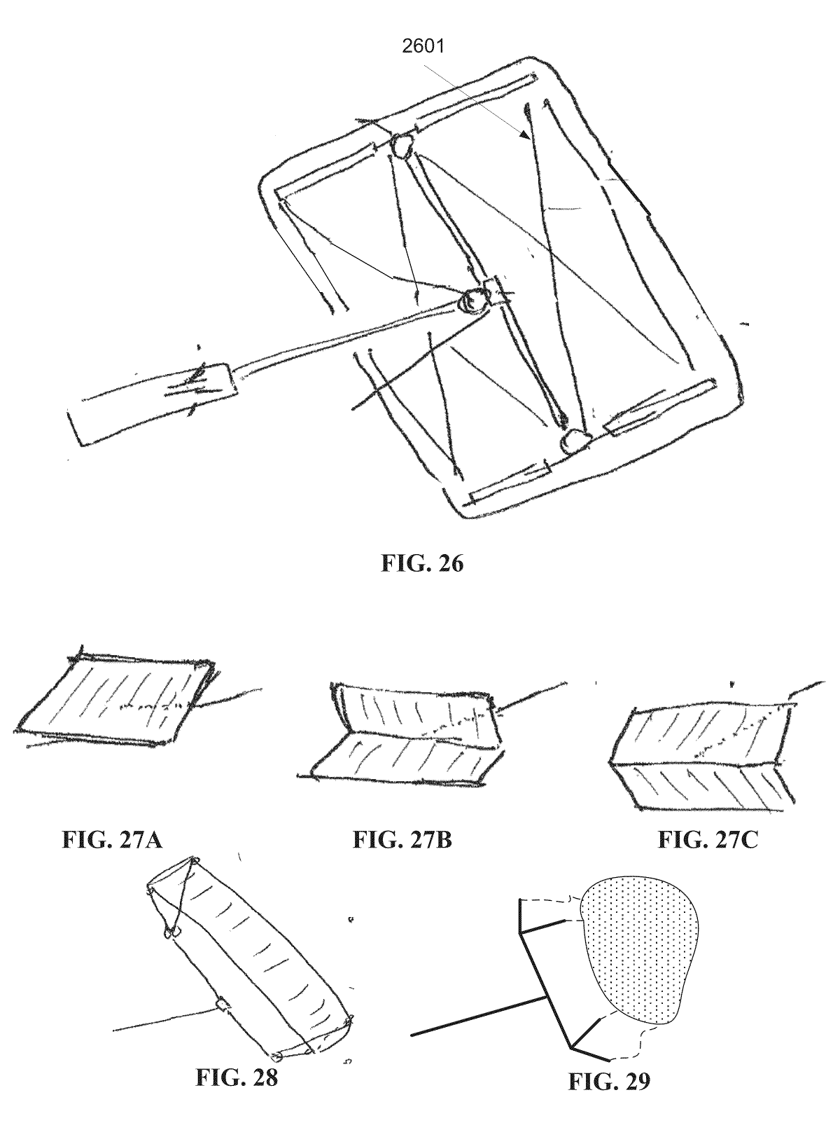

According to some embodiments of the present invention, a system, such as depicted in FIGS. 12A-12G, is used for a displacement or an organ or a portion thereof during a surgery. In such an embodiment, the self-extending elements 615 are connected to a displacement sheet ad not to a surgical mesh. Optionally, in order to allow displacing internal organs after the spreading of the displacement sheet, the elasticity of the self-extending elements 615 is reduced, for example by adjusting the elastic connections 625 to the supporting element 610. Optionally, in order to allow displacing internal organs after the spreading of the displacement sheet, holder element 700 is a spherical joint that is adjusted to maintain the angle of the spread displacement sheet during a displacement operation. Optionally, a locking mechanism is used to lock the spherical joint so that the angle of the spread displacement sheet is fixated. Optionally, as shown at FIG. 26, a plurality of supporting threads, such as 2601, are connected between the self-extending elements 615 and the supporting element 610. Optionally, the self-extending elements 615 are set the spread the displacement sheet in a planner manner, for example as shown at FIG. 27A. In such a manner, the system may be used to displace organs. Optionally, the self-extending elements 615 are set the spread the displacement sheet to form a concave shape, for example as shown at FIG. 27B. Optionally, the self-extending elements 615 are set the spread the displacement sheet to form a convex shape, for example as shown at FIG. 27C. In such a manner, the system may be used as a tissue separator.

According to some embodiments of the present invention, a system, such as depicted in FIGS. 7A-7B, is used for carrying a therapeutic film, such as a haemorrhage treatment film and a separating film, for example SEPRAFILM™, which is applied during abdominal surgery and sticks to internal tissues to separate organs to prevent them from attaching to one another as they heal. In such an embodiment, the film is placed instead of the mesh. In use, the physician pushes the film in a spread state, using the system, toward an intrabody surface and holds it sticks to internal tissues. Now, the guiding rod is retrieved via the channel, for example as described above.

Optionally, the device is adjusted to support rectangular films of size of about 10×13 cm to about 15×15 cm. According to some embodiments of the present invention, the film is rolled in a vertical double cylinder scroll fold together with 6-8 the self-extending elements 400 (FIG. 7A). Optionally, the self-extending elements are in the form of super-elastic bands that are stronger in the vertical axis (with respect to the vertical direction of the double cylinder scroll).

According to some embodiments of the present invention, a device, such as depicted in FIGS. 12A-12G or in FIG. 7A-7B, is used for carrying a supporting sheet which is applied during abdominal surgery to bound turn organs, such as the spleen, the liver, the uterus, a kidney, the heart, a limb, and the like so as to encourage the healing thereof. In such an embodiment, the supporting sheet is placed instead a surgical mesh 10 (for the sake of explanation). In use, the physician pushes the supporting mesh in a spread state, using the device, toward the target organ and releases the supporting mesh to encircle the organ. Now, the guiding rod is retrieved via the channel, for example as described above.

According to some embodiments of the present invention, a device, such as depicted in FIGS. 12A-12G or in FIG. 7A-7B, is used for applying therapeutic and/or diagnostic agents and/or materials on intrabody surfaces. For example, the therapeutic agents may be haemorrhage treatment agents.

In such an embodiment, the self-extending elements 615 are connected to a carrier sheet and not to a surgical mesh. In such an embodiment, the carrier sheet is laminated or otherwise covered with the therapeutic and/or diagnostic agents. Alternatively, the therapeutic and/or diagnostic agents are spread on the carrier sheet and folded with is, for example as described above. In use, the physician pushes the carrier sheet toward an intrabody surface and holds it until some of the therapeutic and/or diagnostic agents are passed into the intrabody surface. Now, the device with the carrier sheet is retrieved via the channel, for example as described above.

According to some embodiments of the present invention, a device, such as depicted in FIGS. 7A-7B or in FIGS. 12A-12G, is used for carrying a graft, for example a flexible vascular graft, such as an aortic root graft. In such an embodiment, the graft is connected to the self expending elements 615 and not a surgical mesh so that the spreading of the self-extending elements 615 spreads the graft. In use, the physician places the graft in a spread state, in a target area, and holds it to allow the attaching thereof, for example by stitching. Now, the guiding rod is retrieved via the channel, for example as described above. For example, FIG. 28 is simplified schematic illustration of an exemplary guide rod 50 with self-extending elements 615 which spread a graft 2604.

According to some embodiments of the present invention, a device, such as depicted in FIGS. 7A-7B or in FIGS. 12A-12G, is used for carrying a pouch, for example a flexible sheet, to collect debris and/or taken off intrabody organs or parts thereof. In such an embodiment, the pouch is connected to the self expending elements 615 and not a surgical mesh so that the spreading of the self-extending elements 615 spreads the pouch. In use, the physician places the pouch, in a spread state, in front of the organ he wants to remove and pull the rod 50 to proximate the removed organ or portion thereof toward the channel. Now, the guiding rod may be retrieved via the channel, with the pouch and its content, optionally after the content has be shredded as known in the art. For example, FIG. 29 is simplified schematic illustration of an exemplary guide rod 50 with self-extending elements 615 which spread an exemplary pouch.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

1. A modular surgical mesh delivery and positioning system comprising:
a prepackaged mesh unit, wherein the prepackaged mesh unit is configured for accommodating meshes of different size and shape, the prepackaged mesh unit including a mesh collapsed with a plurality of self-extending elements held by a guide rod, wherein the number and configuration of the self-extending elements is selected to match the mesh packaged in the prepackaged mesh unit;
a hand guide unit operative to engage onto one end of the guide rod and thereby manipulate movement of the prepackaged mesh unit; and
an operative channel adapted to be partially inserted in-vivo through which the prepackaged mesh unit is delivered,
wherein each of the prepackaged mesh unit, the hand guide unit and the operative channel are separately adaptable to specific applications while still maintaining compatibility with each other.

2. The system according to claim 1, wherein a length of the guide rod is selected to correspond to a length of the mesh packaged in the prepackaged mesh unit.

3. The system according to claim 1, wherein a length of the hand guide unit to be used with the guide rod is selectable on-site.

4. The system according to claim 1, wherein a diameter of the operative channel is adapted to a diameter of the prepackaged mesh unit.

5. The system according to claim 4, wherein the diameter of the operative channel is smaller than a diameter of the prepackaged mesh unit and is adapted to compress the prepackaged mesh unit during delivery.

6. The system according to claim 1, wherein a length of the operative channel is adapted to the length of the prepackaged mesh unit.

7. The system according to claim 6, wherein the operative channel includes extensions for adjusting a length of the operative channel.

8. The system according to claim 1, wherein the hand guide unit is adapted to controllably latch on to and release the guide rod.

9. A prepackaged mesh unit comprising:
a guide rod configured for remotely positioning a spreadable sheet within a body cavity; and
a plurality of self-extending elements collapsed with the spreadable sheet and held at one end by the guide rod, wherein the self-extending elements have an elastic property and store elastic energy while collapsed with the spreadable sheet, wherein the self-extending elements being connected to said spreadable sheet so as to spread said spreadable sheet when said elastic energy being released.

10. The system according to claim 9, wherein, further comprising a releasing mechanism which maintains said spreadable sheet in a collapsed state and releases said spreadable sheet to spread state when said elastic energy being released.

11. The system according to claim 10, wherein said releasing mechanism comprises a pin for holding at least one thread threaded around said spreadable sheet in a collapsed state and for releasing said at least one thread when said elastic energy being released.

12. The system according to claim 9, wherein said plurality of self-extending elements spread said spreadable sheet, when said elastic energy is released, in a distance of at least 3 centimeters from the tip of said guide rod.

13. The system according to claim 9, wherein said self-extending elements are supported by two supporting elements which are set, after said elastic energy is released, to form an angle of at less than 130° therebetween.

14. The system according to claim 9, further comprising an adaptor having a funnel opening and a trocar connection adapted to be partially inserted into a trocar through which the spreadable sheet unit is delivered.

15. (canceled)

16. The system according to claim 9, further comprising a plurality of supporting threads, connected to strengthening said spreadable sheet.

17-25. (canceled)

26. The prepackaged mesh unit according to claim 9, wherein at least one self-extending element is constructed from at least two wires fitted through a rigid tube, wherein the at least two wires are partially exposed over a length to form an elastic joint.

27-29. (canceled)

30. The prepackaged mesh unit according to claim 9, wherein at least a portion of the self-extending elements are connected to each other by a string or wire.

31. (canceled)

32. The prepackaged mesh unit according to claim 9, wherein the spreadable sheet is rolled in a double cylinder scroll in the prepackaged spreadable sheet unit.

33-34. (canceled)

35. The prepackaged mesh unit according to claim 9, wherein at least one of the plurality of self-extending elements is at least partially constructed from shape memory alloy, the shape memory alloy is 0.4-0.5 mm nickel titanium wire.

36-41. (canceled)

42. The prepackaged mesh unit according to claim 9, wherein the spreadable sheet is a horseshoe shaped spreadable sheet shaped with an indented area and wherein two of the elastic self-extending elements are removably connected to the spreadable sheet on either side of the indented area and are adapted to maintain a ‘V’ shaped angle in an unloaded state.

43-71. (canceled)

72. A method for delivering and positioning a surgical mesh in a normal orifice transendoscopic surgical procedure, the method comprising:
connecting a prepackaged mesh unit to an end of a flexible tube of an endoscope, wherein the prepackaged mesh unit includes a mesh collapsed around a plurality of self-extending elements;
guiding the prepackaged mesh unit to a herniated area; and
releasing packaging of the mesh unit, said self-extending elements operative to flatten out the mesh.

73. The method according to claim 72, further comprising centering said prepackaged mesh unit in front of said herniated area before said releasing.

74. The method according to claim 72, comprising:
releasing a first portion of the self-extending elements, the first portion adapted to deploy a first half of the mesh;
centering the mesh with the herniated area while only the first half of the mesh is deployed; and
releasing a second portion of the self-extending elements, the second portion adapted to deploy a second half of the mesh after the centering.

75. The method according to claim 74 comprising fixating the first half of the mesh after the centering and prior to deploying the second half of the mesh.

76. The method according to claim 74, comprising positioning the mesh on the herniated area by sliding the plurality of self-extending elements and mesh against the herniated area.

77. (canceled)
